(19) United States
(12) Patent Application Publication
(10) Pub. No.: US 2007/0238143 A1
(43) Pub. Date: Oct. 11, 2007

(54) Metal ion mediated fluorescence superquenching assays, kits and reagents

(76) Inventors: Wensheng Xia, Santa Fe, NM (US); Frauke Rininsland, Santa Fe, NM (US); Sriram Kumaraswamy, Santa Fe, NM (US); Stuart Kushon, Santa Fe, NM (US); Liangde Lu, Albuquerque, NM (US); Xiaobo Shi, Los Alamos, NM (US); Casey Stankewicz, Santa Fe, NM (US); Shannon Wittenburg, Santa Fe, NM (US); Komandoor Achyuthan, Santa Fe, NM (US); Duncan McBranch, Santa Fe, NM (US); David Whitten, Albuquerque, NM (US)

Correspondence Address: PEACOCK MYERS, P.C., 201 THIRD STREET, N.W., SUITE 1340, ALBUQUERQUE, NM, 87102, US

(21) Appl. No.: 11/219,673

(22) Filed: Sep. 7, 2005

Related U.S. Application Data
(63) Continuation of application No. 11/008,935, filed on Dec. 13, 2004, now abandoned.
(60) Provisional application No. 60/528,792, filed on Dec. 12, 2003.
(60) Provisional application No. 60/550,733, filed on Mar. 8, 2004.
(60) Provisional application No. 60/604,813, filed on Aug. 27, 2004.

(51) Int. Cl. C12Q 1/48; C12Q 1/42; C07K 14/47

(52) U.S. Cl. 435/15; 435/21; 530/409

(57) ABSTRACT

Reagents and assays for kinase, phosphatase and protease enzyme activity which employ metal ion-phosphate ligand specific binding and fluorescent polymer superquenching are described. The assays provide a general platform for the measurement of kinase, phosphatase and protease enzyme activity using peptide and protein substrates. Reagents and assays based on DNA hybridization and reagents and assays for proteins which employ aptamers, antibodies and other ligands are also described.

This application claims the benefit of: U.S. Provisional Patent Application Ser. No. 60/528,792, filed Dec. 12, 2003; U.S. Provisional Patent Application Ser. No. 60/550,733, filed Mar. 8, 2004; and U.S. Provisional Patent Application Ser. No. 60/604,813, filed Aug. 27, 2004. Each of the aforementioned applications is incorporated by reference herein in its entirety.

### BACKGROUND

1. Technical Field

The present application relates generally to reagents, kits and assays for the detection of biological molecules and, in particular, to reagents, kits and assays for the detection of biological molecules which combine metal ion binding and fluorescent polymer superquenching.

2. Background of the Technology

The enzyme linked immunosorbant assay (i.e., ELISA) is the most widely used and accepted technique for identifying the presence and biological activity of a wide range of proteins, antibodies, cells, viruses, etc. An ELISA is a multi-step “sandwich assay” in which the analyte biomolecule is first bound to an antibody attached to a surface. A second antibody then binds to the biomolecule. In some cases, the second antibody is attached to a catalytic enzyme which subsequently “develops” an amplifying reaction. In other cases, this second antibody is biotinylated to bind a third protein (e.g., avidin or streptavidin). This protein is attached either to an enzyme, which creates a chemical cascade for an amplified calorimetric change, or to a fluorophore for fluorescent tagging.

Despite its wide use, there are many disadvantages to ELISA. For example, because the multi-step procedure requires both precise control over reagents and development time, it is time-consuming and prone to “false positives”. Further, careful washing is required to remove nonspecific adsorbed reagents.

Fluorescence resonance energy transfer (i.e., FRET) techniques have been applied to both polymerase chain reaction-based (PCR) gene sequencing and immunoassays. FRET uses homogeneous binding of an analyte biomolecule to activate the fluorescence of a dye that is quenched in the off-state. In a typical example of FRET technology, a fluorescent dye is linked to an antibody (F-Ab), and this diad is bound to an antigen linked to a quencher (Ag-Q). The bound complex (F-Ab:Ag-Q) is quenched (i.e., non-fluorescent) by energy transfer. In the presence of identical analyte antigens which are untethered to Q (Ag), the Ag-Q diads are displaced quantitatively as determined by the equilibrium binding probability determined by the relative concentrations, [Ag-Q]/[Ag]. This limits the FRET technique to a quantitative assay where the antigen is already well-characterized, and the chemistry to link the antigen to Q must be worked out for each new case.

Other FRET substrates and assays are disclosed in U.S. Pat. No. 6,291,201 as well as the following articles: Anne, et al., “High Throughput Fluorogenic Assay for Determination of Botulinum Type B Neurotoxin Protease Activity”, Analytical Biochemistry, 291, 253-261 (2001); Cummings, et. al., A Peptide Based Fluorescence Resonance Energy Transfer Assay for Bacillus Anthracis Lethal Factor Protease”, Proc. Natl. Acad. Scie. 99, 6603-6606 (2002); Mock, et al., “Progress in Rapid Screening of Bacillus Anthracis Lethal Activity Factor”, Proc. Natl. Acad. Sci. 99, 6527-6529 (2002); Sportsman et al., Assay Drug Dev. Technol., 2004, 2, 205; and Rodems et al., Assay Drug Dev. Technol., 2002, 1, 9.

Other assays employing intramolecularly quenched fluorescent substrates are disclosed in the following articles: Zhong, et al., Development of an Internally Quenched Fluorescent Substrate for Escherichia Coli Leader Peptidase”, Analytical Biochemistry 255, 66-73 (1998); Rosse, et al., “Rapid Identification of Substrates for Novel Proteases Using a Combinatorial Peptide Library”, J. Comb. Chem., 2, 461-466 (2000); and Thompson, et al., “A BODIPY Fluorescent Microplate Assay for Measuring Activity of Calpains and Other Proteases”, Analytical Biochemistry, 279, 170-178 (2000).

Assays have also been developed wherein changes in fluorescence polarization have been measured and used to quantify the amount of an analyte. See, for example, Levine, et al., “Measurement of Specific Protease Activity Utilizing Fluorescence Polarization”, Analytical Biochemistry 247, 83-88 (1997). See also Schade, et al., “BODIPY-α-Casein, a pH-Independent Protein Substrate for Protease Assays Using Fluorescence Polarization”, Analytical Biochemistry 243, 1-7 (1996).

There still exists a need, however, to rapidly and accurately detect and quantify biologically relevant molecules such as enzymes and nucleic acids with high sensitivity.

### SUMMARY

According to a first embodiment, a complex is provided which comprises:

a biotinylated polypeptide, wherein the polypeptide comprises one or more phosphate groups; and

a metal cation associated with a phosphate group of the polypeptide.

According to a second embodiment, a method of detecting the presence and/or amount of a kinase or phosphatase enzyme analyte in a sample is provided. The method according to this embodiment comprises:

a) incubating the sample with a biotinylated polypeptide, wherein, for a kinase enzyme analyte, the polypeptide comprises one or more groups which are phosphorylatable by the analyte or, wherein for a phosphatase enzyme analyte, the polypeptide comprises one or more groups which are dephosphorylatable by the analyte;

b) adding to the sample a metal cation, wherein either the metal cation is a quencher or wherein the method further comprises adding to the sample a quencher which can associate with the metal cation;

c) adding to the sample a fluorescer comprising a plurality of fluorescent species associated with one another such that the quencher is capable of amplified superquenching of the fluorescer when the quencher is associated with the fluorescer, wherein the fluorescer is associated with a biotin binding protein; and

d) detecting fluorescence;

wherein the detected fluorescence indicates the presence and/or amount of analyte in the sample.

According to a third embodiment, a method of screening a compound as an inhibitor of kinase or phosphatase enzyme activity is provided. The method according to this embodiment comprises:

a) incubating in a sample a biotinylated polypeptide with a kinase or phosphatase enzyme in the presence of the compound, wherein, for a kinase enzyme assay, the polypeptide comprises one or more groups which are phosphorylatable by the analyte and wherein, for a phosphatase enzyme assay, the polypeptide comprises one or more groups which are dephosphorylatable by the analyte;

b) adding to the sample a metal cation, wherein either the metal cation is a quencher or wherein the method further comprises adding to the sample a quencher which can associate with the metal cation;

c) adding to the sample a fluorescer comprising a plurality of fluorescent species associated with one another such that the quencher is capable of amplified superquenching of the fluorescer when the quencher is associated with the fluorescer, wherein the fluorescer is associated with a biotin binding protein; and

d) detecting fluorescence from the sample in the presence of the compound;

wherein the amount of fluorescence detected in the presence of the compound indicates the inhibitory effect of the compound on kinase or phosphatase enzyme activity.

According to a fourth embodiment, a bioconjugate is provided which comprises:

a polypeptide comprising one or more phosphorylatable or dephosphorylatable groups; and

a quenching moiety conjugated to the polypeptide. The quenching moiety can be rhodamine or another dye with similar spectral characteristics.

According to a fifth embodiment, a bioconjugate as set forth above can further comprise one or more phosphate groups and a cleavage site, wherein the quenching moiety and the phosphate groups are on opposite sides of the cleavage site. Preferably, no phosphate groups are present on the side of the cleavage site to which the quenching moiety is conjugated.

According to a sixth embodiment, a method of detecting the presence and/or amount of a protease enzyme in a sample is provided which comprises:

a) incubating the sample with a bioconjugate comprising a cleavage site and one or more phosphate groups as set forth above, wherein the protease enzyme cleaves the polypeptide at the cleavage site;

b) adding to the sample a fluorescer comprising a plurality of fluorescent species associated with one another such that the quenching moiety is capable of amplified superquenching of the fluorescer when the quenching moiety is associated with the fluorescer, wherein the fluorescer further comprises one or more anionic groups and wherein at least one metal cation is associated with an anionic group of the fluorescer; and

c) detecting fluorescence from the sample;

wherein the detected fluorescence indicates the presence and/or amount of protease enzyme in the sample.

According to a seventh embodiment, a kit for detecting the presence and/or amount of a kinase or protease enzyme analyte in a sample is provided which comprises:

a first component comprising a bioconjugate as set forth above; and

a second component comprising a fluorescer, the fluorescer comprising a plurality of fluorescent species associated with one another such that the quenching moiety of the bioconjugate is capable of amplified superquenching of the fluorescer when the quenching moiety is associated with the fluorescer, wherein the fluorescer further comprises one or more anionic groups and wherein at least one metal cation is associated with an anionic group of the fluorescer.

According to an eighth embodiment, a method of detecting the presence and/or amount of an enzyme analyte in a sample is provided which comprises:

a) incubating the sample with a bioconjugate as set forth above, wherein the polypeptide of the bioconjugate comprises groups which are phosphorylatable or dephosphorylatable by the enzyme analyte;

b) adding to the sample a fluorescer comprising a plurality of fluorescent species associated with one another such that the quenching moiety is capable of amplified superquenching of the fluorescer when the quenching moiety is associated with the fluorescer, wherein the fluorescer further comprises one or more anionic groups and wherein at least one metal cation is associated with an anionic group of the fluorescer; and

c) detecting fluorescence from the sample;

wherein the detected fluorescence indicates the presence and/or amount of analyte in the sample.

According to a ninth embodiment, a kit for detecting the presence of an analyte in a sample is provided which comprises:

a first component comprising a quencher; and

a second component comprising a biotinylated polypeptide, wherein the polypeptide can be modified by the analyte and wherein the polypeptide modified by the analyte associates with the quencher.

According to a tenth embodiment, a method of detecting the presence and/or amount of a phosphodiesterase enzyme in a sample is provided which comprises:

a) incubating the sample with a bioconjugate comprising a quencher conjugated to cyclic AMP or cyclic GMP;

b) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and

c) detecting fluorescence from the sample;

wherein the amount of detected fluorescence indicates the presence and/or amount of phosphodiesterase enzyme in the sample.

According to an eleventh embodiment, a method of detecting kinase enzyme activity of a polypeptide substrate is provided which comprises:

a) incubating the polypeptide substrate and a quencher labeled polypeptide comprising one or more phosphorylatable groups with a sample comprising a kinase enzyme;

b) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and

c) detecting fluorescence from the sample;

wherein phosphorylation of the polypeptide substrate results in an increase in fluorescence; and

wherein the amount of fluorescence detected indicates the presence and/or amount of kinase enzyme activity of the polypeptide substrate.

According to a twelfth embodiment, a method of detecting the presence and/or amount of a nucleic acid analyte in a sample is provided which comprises:

a) incubating the sample with a polynucleotide comprising a quencher conjugated to the polypeptide in a first terminal region of the polynucleotide and a phosphate group in a second terminal region of the polynucleotide, wherein at least a portion of the first and second terminal regions of the polynucleotide can hybridize together to form a hairpin structure and wherein a central region of the polynucleotide between the terminal regions comprises a nucleic acid sequence which can hybridize to the nucleic acid analyte thereby disrupting the hairpin structure and resulting in separation of the quencher and the phosphate group of the polynucleotide;

b) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and

c) detecting fluorescence from the sample;

wherein the detected fluorescence indicates the presence and/or amount of nucleic acid analyte in the sample.

According to a thirteenth embodiment, a method of detecting the presence and/or amount of a nucleic acid analyte in a sample is provided which comprises:

a) labeling nucleic acids in the sample with a quencher;

b) incubating the sample with a polynucleotide comprising a phosphate group in a first terminal region of the polynucleotide, wherein the polynucleotide comprises a nucleic acid sequence which can hybridize to the nucleic acid analyte;

c) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and

d) detecting fluorescence from the sample;

wherein hybridization of the nucleic acid analyte to the polynucleotide results in a decrease in fluorescence; and

wherein decreased fluorescence indicates the presence and/or amount of nucleic acid analyte in the sample.

According to a fourteenth embodiment, a method of detecting the presence and/or amount of a nucleic acid analyte in a sample is provided which comprises:

a) incubating the sample with a first polynucleotide comprising a phosphate group in a terminal region thereof and a second polynucleotide comprising a quencher conjugated to the second polynucleotide in a terminal region thereof, wherein the second polynucleotide and the nucleic acid analyte can hybridize to the first polynucleotide;

b) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and

c) detecting fluorescence from the sample;

wherein hybridization of the nucleic acid analyte to the first polynucleotide results in an increase in fluorescence; and

wherein the amount of fluorescence detected indicates the presence and/or amount of nucleic acid analyte in the sample.

According to a fifteenth embodiment, a method of detecting the presence and/or amount of a polypeptide analyte in a sample is provided which comprises:

a) incubating the sample with: a nucleic acid aptamer comprising a phosphate group in a terminal region thereof, wherein the nucleic acid aptamer can bind to the polypeptide analyte; and a polynucleotide comprising a quencher, wherein the polynucleotide can hybridize to the nucleic acid aptamer;

b) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and

c) detecting fluorescence from the sample;

wherein binding of the polypeptide analyte to the nucleic acid aptamer results in an increase in fluorescence; and

wherein the amount of fluorescence detected indicates the presence and/or amount of polypeptide analyte in the sample.

According to a sixteenth embodiment, a complex is provided which comprises:

a polypeptide comprising a biotin moiety wherein one or more amino acid residues of the polypeptide are phosphorylatable or dephosphorylatable; and

a biotin binding protein conjugated to a quenching moiety;

wherein the biotin moiety of the polypeptide is associated with the biotin binding protein via protein-protein interactions; and

wherein the quenching moiety is capable of amplified super-quenching of a fluorescer when associated therewith.

According to a seventeenth embodiment, a method of detecting the presence and/or amount of a kinase or phosphatase enzyme analyte in a sample is provided which comprises:

a) incubating the sample with a complex as set forth above, wherein for a kinase enzyme analyte, the polypeptide comprises one or more groups which are phosphorylatable by the analyte and, wherein for a phosphatase enzyme analyte, the polypeptide comprises one or more groups which are dephosphorylatable by the analyte;

b) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and

c) detecting fluorescence from the sample;

wherein the amount of fluorescence detected indicates the presence and/or amount of analyte in the sample.

According to a eighteenth embodiment, a method of detecting the presence and/or amount of a kinase or phosphatase enzyme analyte in a sample is provided which comprises:

a) incubating the sample with a biotinylated polypeptide comprising either one or more groups which are phosphorylatable by the analyte for a kinase enzyme analyte assay or one or more groups which are dephosphorylatable by the analyte for a phosphatase enzyme analyte assay;

b) adding to the incubated sample a biotin binding protein conjugated to a quenching moiety;

c) adding to the sample a fluorescer comprising a plurality of fluorescent species associated with one another such that the quenching moiety is capable of amplified superquenching of the fluorescer when the quenching moiety is associated with the fluorescer, wherein the fluorescer further comprises one or more anionic groups and wherein at least one metal cation is associated with an anionic group of the fluorescer; and

d) detecting fluorescence from the sample;

wherein the detected fluorescence indicates the presence and/or amount of analyte in the sample.

### BRIEF DESCRIPTION OF THE DRAWINGS

FIGS. 1A and 1B show the chemical structures of polymers which can be used in metal ion mediated fluorescence superquenching assays.

FIG. 2 is a schematic of an assay for enzyme mediated phosphorylation or dephosphorylation activity based on metal ion mediated fluorescence superquenching.

FIG. 3 is a Stern-Volmer plot for the quenching of a gallium sensor by a Rhodamine labeled phosphorylated peptide.

FIGS. 4A and 4B are graphs showing endpoint and kinetic assays for Protein Kinase A (PKA).

FIG. 5 is a graph showing Protein Kinase A (PKA) assay response in the presence of an inhibitor.

FIG. 6 is a graph demonstrating EC₅₀ and limit of detection for protein tyrosine phosphatase 1B (PTB-1B) phosphatase assay.

FIG. 7 is a graph showing the inhibition of protein tyrosine phosphatase 1B (PTB-1B) activity.

FIG. 8 is a schematic of a protease assay based on metal ion mediated fluorescence superquenching.

FIG. 9 is a schematic of a blocking kinase assay using protein and peptide substrates based on metal ion mediated superquenching.

FIG. 10 is a graph showing a fluorescence turn-on blocking kinase assay using PKCA as an example.

FIG. 11 is a schematic of a phosphodiesterase assay employing metal ion-mediated superquenching.

FIG. 12 is a graph showing the results of monitoring Trypsin activity in a real time or kinetic assay format.

FIG. 13 illustrates the detection of phosphorylated polypeptides according to one embodiment.

FIG. 14 is a graph showing relative fluorescence as a function of protein kinase A (PKA) concentration in an assay using a biotinylated peptide substrate (BT) according to one embodiment.

FIG. 15 is a chart showing the relative fluorescence response to phosphorylated and non-phosphorylated histone.

FIG. 16 is a graph showing relative fluorescence as a function of protein tyrosine phosphatase-1B (PTP-1B) concentration in an assay using a biotinylated peptide substrate (BT) according to a further embodiment.

FIG. 17 illustrates an assay wherein a quencher-tether conjugate (QT) associates with a metal ion and fluorescent polymer ensemble resulting in amplified superquenching of the fluorescent polymer.

FIG. 18 is a graph showing a phosphopeptide calibrator curve for a metal ion mediated superquenching assay.

FIG. 19 shows a Protein Kinase-A concentration curve obtained from a metal ion mediated superquenching assay.

FIG. 20 is a schematic for a kinase enzyme activity sensor based on metal ion mediated fluorescence superquenching via association of a streptavidin quencher molecule added in a second step to kinase reaction.

FIGS. 21A and 21B are graphs comparing endpoint assays for PKA using the two-step approach with biotinylated substrates and a quencher (i.e., Rhodamine) labeled substrate wherein FIG. 21A shows RFU as a function of PKA concentration and FIG. 21B shows % phosphorylation as a function of PKA concentration.

FIG. 22 is a bar chart illustrating the results of a screen using seven (7) different biotinylated peptide substrates which were each reacted with 3 different enzymes (i.e., PTP-1B, PKCα and PKA).

### DETAILED DESCRIPTION

The quencher-tether-ligand (QTL) approach to biosensing takes advantage of superquenching of fluorescent polyelectrolytes by electron and energy transfer quenchers. The QTL assay platform utilizes the light harvesting ability of conjugated polymers along with their highly delocalized excited state to provide amplified fluorescent signal modulation in response to the presence of very small quantities of electron and energy transfer species. This novel technology has been applied to the highly sensitive detection of proteins, small molecules, peptides, proteases and oligonucleotides by associating the signal modulation phenomenon with antigen-receptor, substrate-enzyme and oligonucleotide-oligonucleotide binding interactions. [1-9]

In one approach, the fluorescent polymer, P, is co-located with biotin-binding protein either in solution or on a solid support, and forms an association complex with a quencher-tether-biotin (QTB) bioconjugate through biotin-biotin binding protein interactions. The QTB bioconjugate includes a quencher, Q, linked through a reactive tether to biotin, which strongly binds the biotin binding protein co-located with the polymer, P. The reaction of the QTB bioconjugate with the target analyte modifies the polymer fluorescence in a readily detectable way.

As described herein, an alternate way of associating the QTL bioconjugate with a fluorescent polymer has been developed which uses the self-organizing capability of fluorescent polyelectrolytes either as individual molecules in solution or as an assembly on a support to complex with metal ions. The thus complexed metal ions can associate with selectivity to coordinating groups (e.g., phosphate groups) incorporated into the QTL bioconjugate thus providing the basis for selective detection of, for example, proteins, small molecules, peptides, proteases, kinases, phosphatases and oligonucleotides. [10-11]

The efficiency with which an acceptor molecule (i.e., quencher) can quench the efficiency of a donor molecule is dependent on the distance that separates the two entities. In constructing assays, the tethering of molecules (to bring the acceptor and donor together) can be accomplished by common strategies such as covalent linkage, and the biotin-avidin interaction. Covalent linkage is an excellent approach for resonance energy transfer because it places the quencher directly onto the acceptor making them one molecule. The distance between the two can therefore be as small as a single bond length. The interaction between biotin and a biotin binding protein (BBP) such as avidin, on the other hand, provides extensive versatility because nearly any molecule can be covalently linked to biotin. However, biotin binding proteins are generally larger that 60 kilodaltons, and as a result when the acceptor and donor are brought together through a biotin-BBP interaction, the distance between the acceptor and donor can be significant.

As a general replacement for the biotin-BBP interaction, we have proposed a metal-ion phosphate interaction for the co-location of acceptors and donors in superquenching assays. As with the biotin-BBP interaction this strategy is generally applicable because many molecules can be phosphorylated. In addition, this strategy is a general improvement over the biotin-avidin interaction because the end-to-end distance of the tether (i.e., the coordination distance between the metal ion and the phosphate) is significantly shorter. The affinity of metal ions for ligands such as phosphate groups is significantly lower than that of the biotin-BBP interaction (Kₐ=10⁵⁻⁷ versus 10¹³⁻¹⁵).

According to one embodiment, a novel sensor comprising fluorescent polyelectrolytes either as individual molecules in solution or as an assembly on a support complexed to metal ions is provided. The metal ions of the sensor can further associate with selectivity to ligands (e.g., phosphate groups) incorporated into the QTL bioconjugate and provide the basis for selective detection of the same molecules described above (e.g., proteins, small molecules, peptides, proteases, kinases, phosphatases and oligonucleotides) including, but not limited to, end-point and kinetic modes. As will be developed below, for some assays the coordinating group-metal ion binding provides an alternative to biotin-biotin binding protein association. In other examples the coordinating group is attached or removed from the quencher portion of the QTL so as to provide for a quench, or a recovery (or both) of sensor fluorescence.

Various embodiments described herein employ fluorescent polymer-QTL superquenching and metal ion-phosphate ligand specific binding to provide improved assays for kinase, phosphatase and protease activity. Metal ion mediated superquenching of fluorescent polymers provides a general platform for the measurement of kinase, phosphatase and protease enzyme activity using peptide and protein substrates as well as a more general approach for carrying out assays based on DNA hybridization and assays for proteins employing aptamers, antibodies and other ligands.

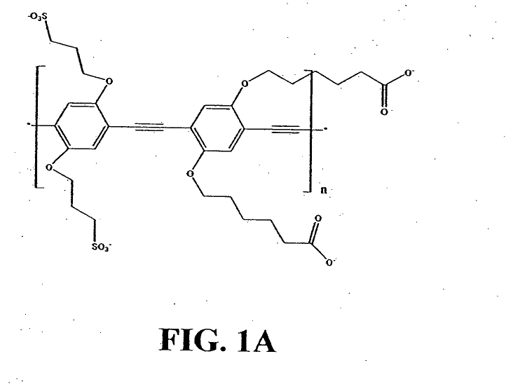

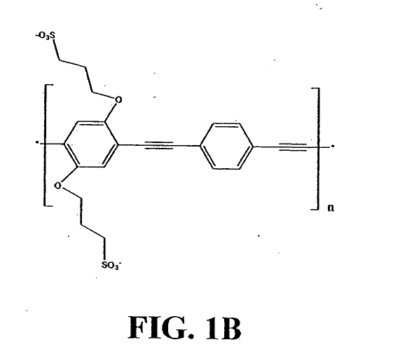

Conjugated polymers in the poly(phenyleneethynylene) (PPE) family can be prepared with a variety of functional groups appended on the aromatic rings. Among the polymers synthesized with pendant anionic groups are those shown in FIGS. 1A and 1B. FIG. 1A shows the molecular structure of sulfo poly p-phenyleneethynylene (PPE-Di-COOH) conjugated polymer. FIG. 1B shows the molecular structure of sulfo poly p-phenyleneethynylene (PPE) conjugated polymer. Both of these polymers can associate with cationic microspheres in water to form stable polymer coatings. The polymer coated microspheres exhibit strong fluorescence. The overall charge on the polymer-coated microspheres can be tuned by varying the degree of polymer loading and by varying the structure of the polymer.

It has been found that fluorescent polymer coated microspheres can associate with metal cations and that the loading of metal cations may depend on the loading level of the polymer on the microsphere. Certain metal ions such as Fe³⁺ and Cu²⁺ can quench the polymer fluorescence while others such as Ga³⁺ do not. In some embodiments, Ga³⁺ is used to mediate superquenching of microsphere-bound polymer fluorescence under conditions where, in the absence of the metal ions, little or no quenching would occur.

For example, a phosphorylated peptide containing a dye:

| | |
|---|---|
| Rhodamine-LRRA(pS)LG | SEQ ID NO:1 |

wherein pS designates phosphorylated serine, which should serve as a good energy transfer quencher for the polymer was found to have little or no quenching of the fluorescence of polymer-coated microspheres. After the polymer-coated microspheres are “charged” by the addition of Ga³⁺, however, addition of the same peptide to the suspensions results in a pronounced quenching of the polymer fluorescence. In contrast, peptides containing only a phosphorylated residue or only the quencher dye, such as the peptide represented by:

| | |
|---|---|
| Rhodamine-LRRASLG | SEQ ID NO:2 |

produce little effect on the polymer fluorescence under the same conditions. The specific association of a phosphorylated biomolecule with the metal ion charged polymer can be the basis of a number of assays as described below.

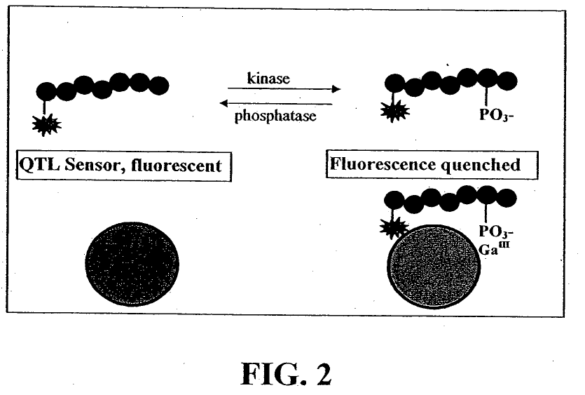

FIG. 2 shows schematically a sensor based on metal ion mediated superquenching which can be used in kinase or phosphatase activity assays. FIG. 2 shows how the phosphorylation or dephosphorylation of rhodamine peptide substrates by target enzymes can be detected by the addition of the QTL sensor. The peptide products are labeled with a rhodamine quencher and brought to the surface of the polymer by virtue of specific phosphate binding to the Ga³⁺ metal ion. The resulting quench of polymer fluorescence is concomitant with phosphorylation or dephosphorylation of the polypeptide substrate. This type of assay can be used for enzymes which moderate phosphorylation or dephosphorylation for biologicqal substrates including, but not limited to, peptides, proteins, lipids, carbohydrates and nucleotides or small molecules.

Kinase/Phosphatase Assays

Phosphorylation and dephosphorylation of proteins mediate the regulation of cellular metabolism, growth, differentiation and cell proliferation. Aberration in enzymatic function can lead to diseases such as cancer and inflammation. More than 500 kinases and phosphatases are thought to be involved in the regulation of cellular activity and many among them are targets for drug therapy.

Protein Kinase A (PKA) is a cAMP dependent protein kinase and functions as an effector of many cAMP-elevating first messengers such as hormones and neurotransmitters. The ubiquitous distribution of PKA and it's flexible substrate recognition properties make PKA a central element in many processes of living cells, such as in the inhibition of lymphocyte cell proliferation and immune response, mediation of long-term depression in the hippocampus and sensory nerve transmission. Protein Tyrosine Phosphatase-1B (PTP-1B) has recently been shown to be a negative regulator of the insulin signaling pathway suggesting that inhibitors to PTP-1B might be beneficial in the treatment of type 2 diabetes.

Of the kinases, 90% phosphorylate serine residues, 10% phosphorylate threonine residues and 0.1% phosphorylate tyrosine residues. Although it has become possible to develop anti-phosphotyrosine antibodies, antibodies against phospho-serine and threonine residues are of low affinity and often specific to only one kinase. Currently, non-antibody-based high-throughput screening (HTS) assays are based on methods such as time-resolved fluorescence (TRF), fluorescence polarization assays (FP) or fluorescence resonance energy transfer (FRET). These assays require specialized equipment and/or suffer from low fluorescence intensity change as a function of enzyme activity.

We sought to enhance sensitivity in the measurement of enzymatic activity by amplifying the fluorescence signal using superquenching as described above. The sensor platform can comprise a modified anionic polyelectrolyte fluorescer such as the poly(phenylenethylene) (PPE) derivative shown in FIG. 1A. The PPE fluorescer can be immobilized by adsorption on positively charged microspheres. This polymer exhibits photoluminescence with high quantum efficiency and has been used for detection of protease activity. [9] In this platform, a reactive peptide sequence was used which is flanked by a N-terminal quencher and a C-terminal biotin. The peptide binds to PPE coated microspheres that are co-located with biotin binding proteins, resulting in a near total quenching of PPE fluorescence. Enzyme mediated cleavage of the peptide leads to a reversal of fluorescence quenching that was linear with enzymatic activity. It has been demonstrated that a single energy acceptor dye can quench the photoluminescence from approximately 49 repeat units per quencher. [9]

Fluorescent polymer superquenching can be adapted to the biodetection of kinase/phosphatase enzyme activity as illustrated in FIG. 2. As shown in FIG. 2, multivalent metal ions can strongly associate with anionic conjugated polymers in solution, resulting in modification and/or quenching of polymer fluorescence. Since the overall charge on a polymer-microsphere ensemble can be tuned, these ensembles can afford a platform whereby metal ions associate with the polymer without strongly quenching the polymer fluorescence while retaining the ability to complex with specific ligands. The approach is similar to that used in immobilized metal ion affinity chromatography (IMAC) whereby metal ions can specifically trap phosphorylated compounds by coordination with the phosphate oxygen at low pH. See, for example, Morgan et al., Assay Drug Dev. Technol., 2004, 2, 171.

As described herein, gallium can associate with fluorescers (including, but not limited to, anionic conjugated polymers such as those shown in FIGS. 1A and 1B and other fluorescers comprising a plurality of fluorescent species) without quenching the polymer emission. The gallium can exist as monomeric Ga³⁺ or as a multimeric ensemble such as a polyoxo species. The fluorescer-associated gallium can also associate with phosphorylated peptides such that, when the peptide contains a dye such as rhodamine, metal ion mediated polymer superquenching occurs. The fluorescer can be associated with a surface of a solid support such as a microsphere. This approach provides the basis for a sensitive and selective kinase/phosphatase assay as illustrated in FIG. 2.

In the case of the fluorescence quench (turn off) kinase assay, the quench of polymer fluorescence is linear with enzyme activity. As described in the following example, the assay can be carried out a near physiological pH and allows flexibility in constructing real time or end point assays. The assays are instantaneous, “mix and read” and require no wash steps or complex sample preparation.

Example 1 below shows robust assays for protein kinase A (PKA) and protein tyrosine phosphatase 1B (PTB-1B) enzyme activities. The assays routinely deliver Z′ values greater than 0.9 at substrate conversion of 10-20%. In the example shown below, the kinase assay provides fluorescence signal attenuation as a function of enzyme activity while the phosphatase assay provides signal enhancement with increasing enzyme activity. Since, for peptides such as SEQ ID NO: 1, the quencher may exhibit sensitized fluorescence as a consequence of the quenching of polymer fluorescence, the assays can exhibit signal enhancement or reduction in the same sample, depending on the wavelengths monitored. Accordingly, ratiometric measurements can be made. Additionally, detection can be carried out by monitoring fluorescence polarization in the quencher of the peptide. For protein kinase, phosphatase and protease assays based on metal ion mediated superquenching, both end point and kinetic assays may be carried out.

### EXAMPLE 1

### Assays for Protein Kinase a (PKA) and Tyrosine Phosphatase Activity 1B (PTP-1B)

The following peptides were used as enzyme substrates and as phospho-peptide calibrators.

For detection of PKA activity:

| | |
|---|---|
| Rhodamine-LRRASLG | SEQ ID NO:2 |

and the calibrator peptide:

| | |
|---|---|
| Rhodamine-LRRA(pS)LG | SEQ ID NO:1 |

were synthesized by Anaspec.

For detection of phosphatase activity:

| | |
|---|---|
| Rhodamine-KVEKIGEGT(pY)GVVYK | SEQ ID NO:3 |

and the calibrator peptide:

| | |
|---|---|
| Rhodamine-KVEKIGEGTYGVVYK | SEQ ID NO:4 |

were synthesized by American Peptide Company.

Recombinant PKA was purchased from Promega. Enzyme PTP-1B as well as inhibitor RK682 were purchased from Biomol. A Staurosporine inhibitor for PKA was purchased from Sigma. Polystyrene amine functionalized beads were obtained from Interfacial Dynamics.

The performance of sensor beads was determined by adding 15 μL of a 1 μM peptide solution (either rhodamine-phospho-peptide or rhodamine-non-phospho-peptide) in assay buffer to 15 μL of sensor in a detector buffer. The fluorescence of the mixture was measured using a SpectraMax Gemini XS plate reader (Molecular Devices, Inc.) in well scan mode and with excitation at 450 nm with a 475 nm cutoff filter and emission at 490 nm.

The polymer whose structure is shown in FIG. 1A was chosen as a sensor for kinase/phosphatase assays based upon the discovery that di- or trivalent metal ions can strongly associate with anionic polymers such as those shown in FIGS. 1A and 1B in solution. No quench of emission was observed when GaCl₃ in a concentration of 340 μM was added to a solution comprising microspheres coated with PPE-Di-COOH. At higher concentrations of GaCi₃, quenching of fluorescent emissions was observed. However, when using an optimal concentration of Ga³⁺, it was found that rhodamine labeled phospho-peptides provided a strong quench of polymer fluorescence whereas little modulation of fluorescence was observed when non phosphorylated rhodamine labeled peptides were used.

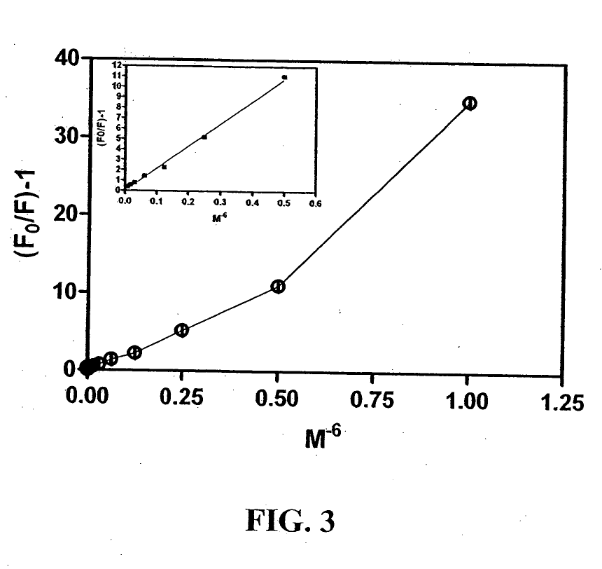

FIG. 3 shows a Stem Volmer plot obtained for Rhodamine labeled PTP-1B phosphopeptide substrate. The Stem Volmer constant (Kₛᵥ) provides a quantitative measure of quenching where F₀ is the intensity of fluorescence in the absence of quencher and F the fluorescence intensity in the presence of quencher. The Kₛᵥ determined here is relatively large (i.e., 2×10⁷ M⁻¹). The 50% quench gives (PRU/Q)50=50, demonstrating the occurrence of superquenching.

As shown above, assays have been developed using quencher labeled substrates. Upon phosphorylation of the substrate, the peptide associates to the sensor via the phosphate groups and quenches fluorescence. Since the metal-ion coordinating groups specifically bind to phosphates, phosphorylated serine, threonine or tyrosine residues can be detected.

Fluorescent superquenching-based assays for serine and tyrosine enzymes, namely Protein Kinase A (PKA), and Protein Tyrosine Phosphatase 1-B (PTP-1B) are described below.

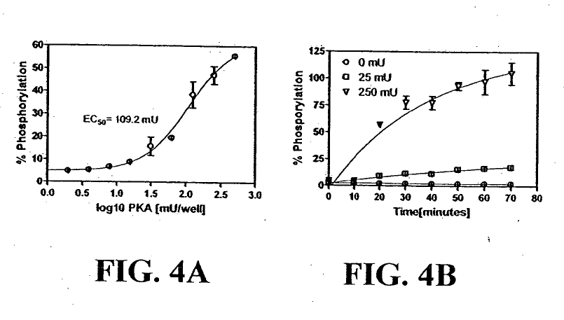

FIG. 4A shows an endpoint measurement of PKA enzyme activity in which an increase in polymer quench correlates with enzyme concentration. Unlike Fe³⁺ coordination assays, which require very low pH, this platform is functional at near physiological pH and thus allows researchers the flexibility of choice in performing real time assays or endpoint assays. A real time assay, that includes the detector mix as part of the enzymatic reaction mix requires approximately 10 fold higher concentrations of enzyme for 50% substrate phosphorylation than an endpoint assay which is shown in FIG. 4B.

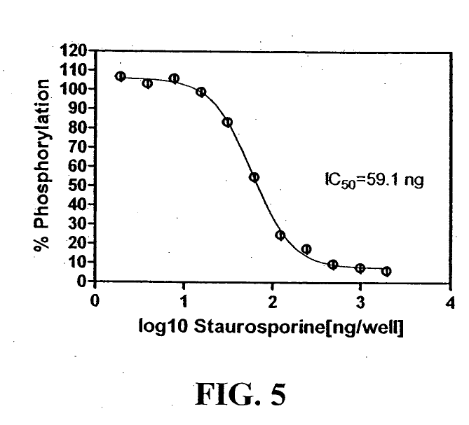

The sensitivity of the assay was tested by using a known inhibitor of PKA activity, Staurosporine. The results are shown in FIG. 5. As shown in FIG. 5, the IC₅₀ obtained using 1 μM substrate in a reaction with 6.5 μM ATP and 200 mU PKA was 59 mU and is in agreement with published values (18.4 mU).

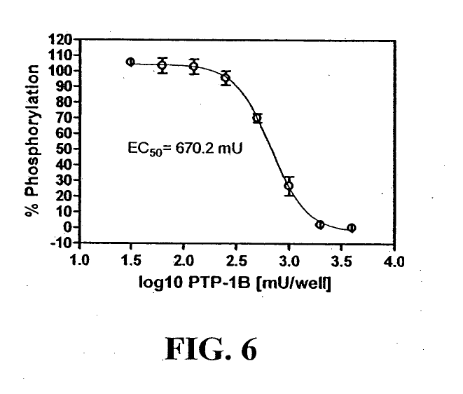

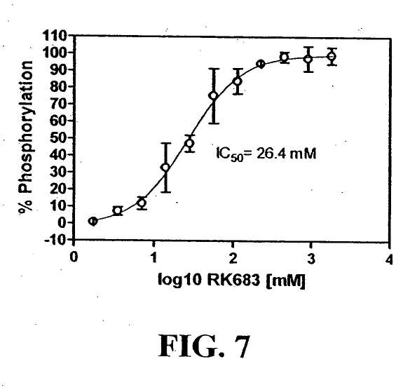

The format was tested for detection of protein tyrosine phosphatase activity 1B (PTP-1B) on a peptide substrate of different length and sequence composition than the one used for PKA. FIG. 6 shows results of EC₅₀ and LOD of enzyme concentration curves measured as endpoint assays or in realtime using PTP-1B on 125 nM substrate. An inhibitor curve using the known inhibitor RK-682 yields an excellent IC₅₀ of 26.4 nM.

The statistical parameters that can be delivered with this assay were determined by evaluating known amounts of phospho peptide calibrator peptide in replicates of 8 (FIG. 6). The data are excellent and show that this assay is suitable to determine as little as 5-10% substrate conversion with Z′ factors of 0.8 and 0.9 respectively.

The performance of this PKA assay has been compared with a commercially available FRET assay, an ATP consumption assay and an IMAC-based assay. All assays were performed to produce optimal performance in an enzyme concentration curve and where possible using the identical peptide. The IMAC-based assay delivers the lowest sensitivity in an enzyme concentration curve (1 ng compared to 20 pg). In this assay, which is closest to the QTL Lightspeed™ assay in principle, the sensor to detector follows a 1:1 ratio as opposed to the 1:50 ratio in the present format. These results clearly demonstrate the enhanced sensitivity obtainable with superquenching.

Additional assays have been developed using substrates for Akt-1 and PKCα. No significant dependency of fluorescence quench on substrate length or peptide sequence content was observed when using these different substrates. In this regard, the metal ion mediated superquenching assay can be considered generic and offers a major advantage over FRET peptides in which quenching is highly dependent on the distance between the donor and acceptor.

Protease Assays

Protease enzymes cleave amide bonds on their substrate. The use of peptide or protein substrates that contain a quencher and a phosphate group on either side of the cleavage site along with the metal ion-fluorescent polymer ensemble affords the development of highly sensitive assays for the detection of protease enzyme activity.

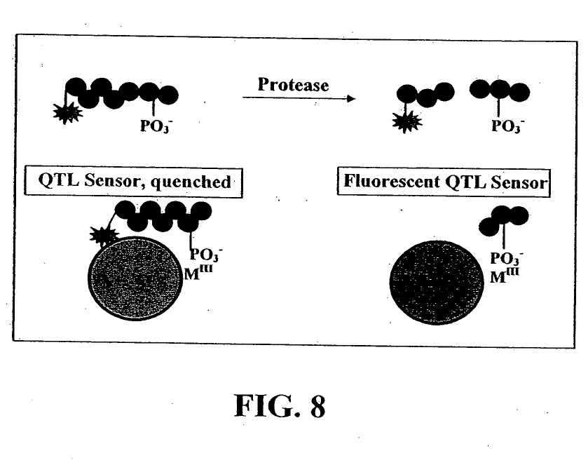

One embodiment of a protease assay is illustrated in FIG. 8. As shown in FIG. 8, when the intact substrate binds the sensor, the sensor fluorescence is quenched by the promixity of the quencher dye. Cleavage of the substrate by the enzyme into fragments separates the quencher from the phosphate group resulting in separation of the quencher and polymer. This separation leads to reduced quench of polymer fluorescence (i.e., enhanced signal from the sensor) in the presence of enzyme activity.

Protease activity can be monitored either real-time or at the end-point in homogeneous or heterogeneous formats. In a homogeneous real-time assay, the substrate can reside on the surface of the polymer-microsphere ensemble. In a homogeneous end-point assay, the substrate and the enzyme can react in solution and, at the end of a specified incubation period, the sensor can be added to the sample to stop the reaction. Protease activity can be monitored ratiometrically when a fluorescent dye is used as the quencher. In a heterogeneous end-point format, biotinylated substrates can be used which contain phosphate groups and a quencher on the same side of a cleavage site. Following cleavage, the peptide species are separated by binding of the biotin species whereas the quencher-labeled portion is transferred and can thereby quench the fluorescer.

### EXAMPLE 2

### A Protease Assay Based on Metal Ion Mediated Fluorescence Superquenching

The peptide substrate for trypsin in this assay is

| | |
|---|---|
| Rhodamine-LRRApSLG. | (SEQ ID NO:1) |

Trypsin cleaves the peptide at the two arginines. The assay performed in this example used the following parameters:

Microsphere-Fluorescer-Gallium ensemble (QTL sensor);

3 μM final Rh-LRRApSLG (SEQ ID NO: 1);

1 U/μL trypsin;

40×10⁶ microspheres (MS)/15 μL;

λₑₓ 430;

λₑₘ 490; and

λ_{co} 475 nm.

The assay was conducted for 1 hr at approximately 22° C. in a 384-well white plate.

The results of this assay are shown below in Table 1.

TABLE 1
Results of Protease Assay Based on Metal
Ion Mediated Fluorescence Superquenching

| | |
|---|---|
| QTL Sensor alone | 88842 |
| No enzyme control | 7771 |
| Sample | 42138 |
| Signal Increase | 34367 |
| Signal/Background | 5.42 |
| Z′ | 0.68 |
| Signal/Noise | 9.69 |

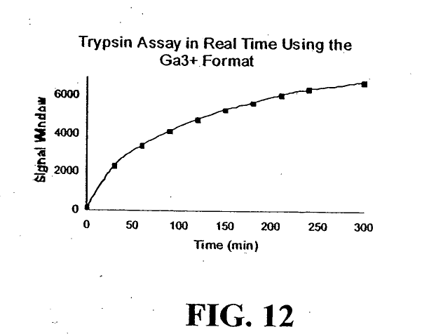

FIG. 12 is a graph showing the results of monitoring Trypsin activity in a “real time” (i.e., kinetic) assay format. As can be seen from FIG. 12, there is a time-dependent increase in Trypsin activity. Correspondingly, the fluorescence signal enhancement occurs with time.

Blocking Assays Using Unlabeled Peptides and Proteins

The basis for the assays described above and shown in FIG. 2 can be adapted to a blocking assay in which a “generic” phosphorylated dye labeled peptide or other substrate containing both a dye and a metal ion binding phosphate (e.g., gallium) quenches the polymer beads containing fluorescent polymer and metal ion in the absence of additional phosphorylated substrates but is “blocked” when a peptide or protein substrate is phosphorylated.

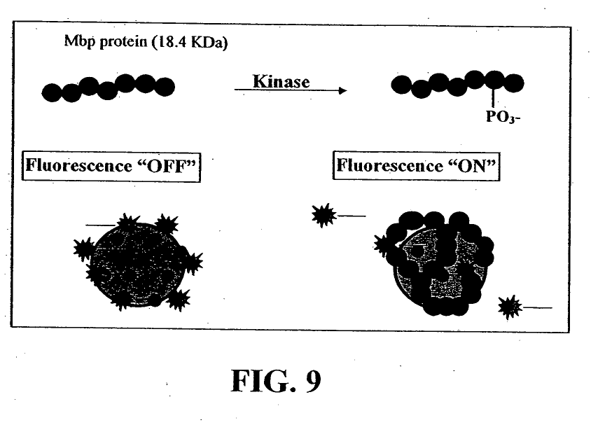

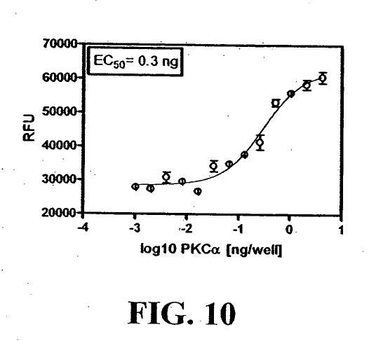

The principle of the assay is shown in FIG. 9 which illustrates schematically a blocking kinase assay based on metal ion mediated superquenching. The assay is most conveniently carried out by adding the sensor to a mixture of enzyme and analyte following incubation for reaction. Any phosphorylated analyte will associate with the sensor as demonstrated in FIG. 9, without quenching the polymer fluorescence. Addition of the “generic” phosphorylated dye labeled peptide will result in a quenching of the polymer fluorescence, limited by the extent of “free” phosphate binding sites on the “blocked” microspheres. The assay functions as a fluorescence “turn-on” assay and offers the additional advantage that no prior derivitization of the substrate need to be done in developing the assay. FIG. 10 shows experimental data for a blocking assay (“fluorescence turn-on”) for PKCa with Myelin Basic Protein (MBP).

The detection of kinase activity on natural protein substrates has several advantages over using peptide substrates as set forth below.

Of the 518 known human kinases (or 2500 isoforms), peptide substrates have been established for only approximately 50 kinases but the target proteins are identified in most cases. Some enzymes may require non-continuous amino acids of a target for effective substrate recognition, binding and phosphorylation, in which case an artificial peptide sequence can not be constructed even if the involved amino acids are identified.

The phosphorylation of natural target proteins is expected to be much more efficient than phosphorylation of peptide substrates. This is important for purpose of cost (of peptide substrates) but also makes identification of inhibitors in HTS more accurate.

The phosphorylation of natural target proteins is more specific than the phosphorylation of artificial substrates. Future attempts to dissect kinase activity in cells will be impeded by the cross recognition of peptide substrates but should work on protein substrates.

Current non-radioactive and non-antibody based assays that allow for detection of phosphorylation of proteins are based on ATP consumption by secondary enzyme Luciferase. Such assays are prone to false negative results in inhibitor screens, as a result of inhibition of the secondary enzyme, Luciferase. FP assays require a large change in molecular motion to obtain a signal, therefore only proteins of small molecular weight can be detected.

### EXAMPLE 3

Phosphorylation of myelin basic protein (MBP) by kinase PKCa was performed in a standard reaction and QTL sensors as described above in Example 2 were added. Phosphorylated MBP binds to the QTL sensor by virtue of specific phosphate binding to the metal coordinating ions and inhibits association of dye-labeled phospho peptide (tracer) in a concentration dependent manner. The resulting fluorescence correlates with the extent of mbp phosphorylation.

This principle is demonstrated in the following example. A concentration of 1 μg mbp was phosphorylated using serially diluted kinase PKCα enzyme for 1 hour at room temperature in a white 384-well Optiplate. Following incubation, 50×10⁶ QTL Sensor beads were added for 10 minutes at approximately 22° C. and subsequently 1 μM dye labeled peptide tracer added. Plates were incubated for 30 minutes at approximately 22° C. and the fluorescence signal monitored using excitation at 450 nm, emission at 490 nm with a 475 nm cutoff filter in a Gemini XS Plate reader (Molecular Devices, Inc.). The fluorescence “turn on” is shown schematically in FIG. 9.

Phosphodiesterase Enzyme Activity Monitored by Metal Ion Mediated Fluorescence Superquenching

The 3′,5′-cyclic nucleotide phosphodiesterases (PDEs) comprise a family of metallophosphohydrolases that specifically cleave the 3′ bond of cyclic adenosine monophosphate (cAMP) and/or cyclic guanosine monophosphate (cGMP) to produce the corresponding 5′-nucleotide. Eleven families of PDEs with varying selectivities for cAMP and cGMP have been identified in mammalian tissues.

PDEs are essential modulators of cellular cAMP and/or cGMP levels. Cyclic-AMP or cGMP are intracellular second messengers that play crucial roles in intracellular signal transduction involved in important cellular processes. PDEs have been targets for drug discovery to treat a variety of diseases. For example, Sidenafil, a selective inhibitor of PDE 5, has been commercialized as a drug (i.e., Viagra®, a registered trademark of Pfizer, Inc.). Several PDE 4 inhibitors are in clinical trials as anti-inflammatory drugs treating diseases such as asthma.

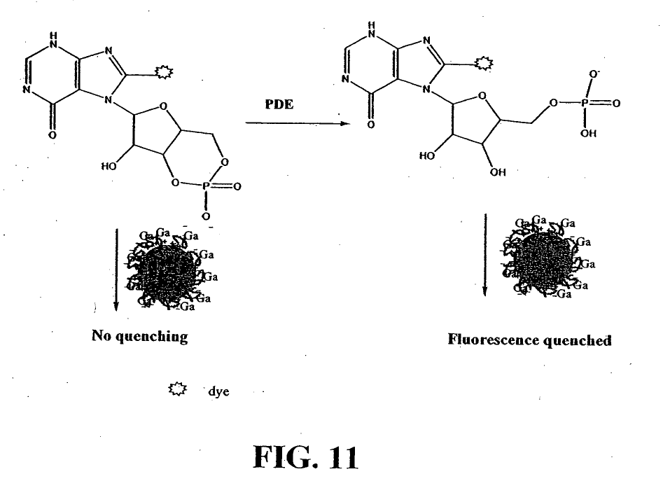

As described above, the QTL sensor shows a high binding affinity towards phosphate groups as demonstrated in the kinase and phosphatase assays. The PDE assay uses a dye-labeled cAMP or cGMP as a substrate to assay the activity of the phosphodiesterase. Dyes including, but not limited to, rhodamine, azo or fluorescein can be coupled to cAMP or cGMP without inhibiting reactivity towards PDEs. Since cAMP or cGMP exists as a phosphodiester, which does not bind strongly to the gallium-polymer surface, there is little initial quenching of the polymer fluorescence. During hydrolysis catalyzed by the PDE, the phosphodiester on these substrates is converted to a phosphate group. The dye then is brought to the vicinity of the microsphere surface through gallium-phosphate specific interactions, resulting in quenching of the polymer fluorescence. FIG. 11 is a schematic depicting a phosphodiesterase assay.

Nucleic Acid Assays

The metal-phosphate mediated binding can be used to generate superquenching assays for DNA and RNA detection. A number of different approaches based on hybridization of a nucleic acid species to a target nucleic acid species which can be in solution or immobilized on a solid support can be used. A first approach utilizes an oligonucleotide that is phosphorylated at one of its termini. The phosphate allows for metal-phosphate mediated co-location of the DNA strand with the conjugated fluorescent polymer. If a phosphate group is attached to the 5-terminus of the oligonucleotide, a complementary target bearing a quencher at the 3′-terminus can be hybridized to the phosphorylated strand. The termini can also be reversed while retaining a functional system. In this hybridized conformation, the quencher would be oriented towards the conjugated polymer to facilitate superquenching. Hence, in the presence of the quencher labeled target, the fluorescence of the polymer is quenched. Such a system can be easily envisioned as an assay for unlabeled DNA by allowing unlabeled and labeled DNA strands to compete for binding to their phosphorylated complementary strand.

A second approach follows a strategy that is similar to the approach used by molecular beacons. A hairpin oligonucleotide bearing a phosphate at one of its termini and a quencher at another can be designed so that the terminal regions of the oligonucleotide are complementary to each other and form a hybridized stem, while the central region of the oligonucleotide is complementary to a target oligonucleotide and forms a single stranded loop when no target is present. Such an oligonucleotide will form a “hairpin” structure which brings the phosphate and the quencher into close proximity by virtue of stem hybridization. When the phosphorylated hairpin oligonucleotide is bound to the metal-polymer complex by virtue of the phosphate metal interaction, a quench will be induced because of the orientation of the quencher towards the polymer. If the phosphate/quencher functionalized oligonucleotide is hybridized to a target that binds to the loop region of the hairpin, the loop region becomes a rigid rod which disrupts the secondary structure of the stem region. This would cause the acceptor and donor pair to be forced apart thereby reducing the quenching of the polymer.

Direct assays for proteins and other targets can also be conducted through a number of routes using the binding properties of DNA aptamers. A phosphorylated DNA aptamer can be bound to the surface of a metal-coated conjugated polymer surface. In the presence of the target molecule (small molecules in size, up to proteins in size) the aptamer conformation of the oligonucleotide should be stabilized (lower ΔG). In the absence of its selected target, the aptamer strand may bear a weak self-structure. If the self-structure of the aptamer can be penetrated by a complementary oligonucleotide that is labeled with a quencher, an assay can be generated. In such an assay, when the aptamer's target is absent, the complementary oligonucleotide-quencher may hybridize to the aptamer. This hybrid can be of the form listed above (i.e., phosphate at 5′-terminus, and quencher at 3′-terminus; or vice-versa), thus the quencher will be oriented to quench the conjugated polymer. In the presence of the aptamer's target, the aptamer self-structure will be stabilized and the oligonucleotide quencher will not be able to hybridize to the aptamer. Hence, in the presence of the aptamer's target, the polymer will fluoresce and in the absence of the aptamer's target the fluorescence will be quenched.

General Phosphate Modification or Consumption

In any system containing a phosphate tethered through any means to a quencher, the modification of the phosphate through chemical means can convert the phosphate to another functionality thus preventing phosphate-metal mediated binding to the metal-polymer complex. Likewise, the binding of the phosphate to other elements may prevent the binding of that same phosphate to a metal polymer complex. In these cases, the quencher will not be co-located with the conjugated polymer and fluorescence will be present. As a general example, complex A, which contains a phosphate tethered through any means to a quencher, can quench the metal polymer complex. If present with a molecule B which bears an affinity for complex A and which also contains elements which will either chemically modify or bind to the phosphate contained in complex A, complex A will not be capable of binding and thereby quenching the metal polymer complex.

Assays, Reagents and Kits Employing Biotin-Tether (BT) Conjugates

According to one embodiment, a kit for conducting an assay for a target analyte is provided. The kit comprises two separate components: a quencher (Q) and a biotin-tether conjugate (BT). The tether (T) of the BT conjugate can comprise, for example, a protein or polypeptide substrate. According to this embodiment, the tether acquires the capacity to associate with the quencher upon interaction with and modification by the target analyte to form a modified tether (T′). Following modification of the tether, a QT′B bioconjugate is formed as a result of the interaction of the BT conjugate with the target analyte followed by association of the modified BT conjugate (BT′) with the quencher (Q). The kit may also comprise a fluorescer component (P). The fluorescer component comprises a plurality of fluorescent species associated in such a manner that the quencher is capable of amplified superquenching of the fluorescer when associated therewith. The fluorescer can be a fluorescent polymer. The fluorescer can be associated with a solid support such as a microsphere, bead or nanoparticle. The solid support can also comprise a biotin binding protein such that interaction of the biotin moiety on the QT′B complex with the biotin binding protein on the solid support results in quenching of fluorecence.

As set forth above, the tether of the BT conjugate can be recognized and modified by association or reaction to the target analyte to form the BT′ conjugate. Modification of the tether renders the modified BT conjugate (BT′) capable of binding the quencher (Q) to form the QT′B complex. This sequence of events can be followed by a modulation of the polymer fluorescence. In particular, a change in fluorescence can be used to indicate the presence and/or the amount of a target analyte in a sample. Moreover, in the absence of a specific association or reaction of the BT conjugate with an enzyme or other target analyte, the fluorescence of P is unaffected by association to the BT conjugate. Accordingly, methods of using a quencher (Q) and a biotin-tether conjugate (BT) as set forth above to determine the presence and/or amount of a target analyte in a sample are also provided.

According to one embodiment, the interaction of the tether (T) of the BT conjugate with a target analyte may result in the removal of a quencher-binding component on the tether. In this embodiment, the capacity of the BT conjugate to bind the quencher (Q) is eliminated as a result of the interaction with the analyte to form the modified conjugate (BT′). Again, this sequence of events can be followed quantitatively via the modulation of polymer fluorescence. In certain embodiments, the reaction of BT and the target analyte may be catalytic, resulting in an amplified modulation of polymer fluorescence.

According to a further embodiment, polymer superquenching may be mediated by a metal-ion. According to this embodiment, a QT conjugate (wherein Q is an electron or energy transfer quencher and T is a reactive tether) can react with a target analyte to introduce, modify or remove a functional group on the tether. The functional group can be a functional group which is capable of associating with a metal ion associated to or co-located (e.g., on a surface of a solid support) with a fluorescent polymer. The modified QT conjugate (QT′) is therefore capable of associating with the ensemble comprising the fluorescent polymer and the metal ion. Consequently, modification of the tether results in a change in the polymer fluorescence. This method may be employed in highly sensitive assays for kinase, phosphatase and other enzymes as target analytes.

Modifiable Tether-Based QTB Approach for the Biodetection of Post-Translational Modification Events

This approach employs a synthetic biotinylated peptide substrate or tether (hereinafter referred to as a “BT conjugate”) which upon interaction with a target analyte is modified to form a BT′ conjugate. In one embodiment, the BT conjugate is incapable of complexing to the non-fluorescent quencher (Q) whereas the modified conjugate (BT′) readily binds to the quencher. This type of interaction leads to a fluorescence “turn-off” assay where the polymer fluorescence decreases with increasing substrate conversion.

In another embodiment, the BT conjugate can readily associate with the dark quencher. However, the BT conjugate loses the ability to associate after interaction with the target analyte to form the modified conjugate (BT′). This type of interaction results in a fluorescence “turn-on” assay.

In a further embodiment, the quencher in the above embodiments can also be a fluorescent moiety. The use of a fluorescent moiety as a quencher can provide sensitized emission of fluorescence. In all of these embodiments, the QTB bioconjugate can form a complex with the polymer-receptor ensemble to modulate the polymer fluorescence efficiently by the superquenching process.

The quencher moiety used in the assay for post-translational modification interaction combines the properties of association to the functional group that is modified on the substrate and amplified superquenching of the fluorescence of the conjugated polymer when present in close proximity. In one embodiment, the quencher can be a transition metal or an organometallic species such as an iron (III) iminodiacetic acid (IDA) type chelate, wherein the ferric iron can both associate strongly to a phosphopeptide and superquench the fluorescent polymer by electron transfer. In another embodiment, the quencher may consist of two distinct moieties, one that promotes association of the quencher to the modified functional group and another that causes polymer quench by energy transfer.

The sensor can comprise a conjugated fluorescent polymer that is co-located with biotin binding protein either on a solid support or in solution. The polymer can be a charged polymer, a neutral polymer, or a “virtual” polymer composed of fluorescent dyes assembled on a non-conjugated backbone or on an oppositely charged surface of a solid support such as a bead or nanoparticle.

Modifiable Tether-Based (QT′B) Approach for Biodetection and Bioassay of Kinase and Phosphatase Enzymes

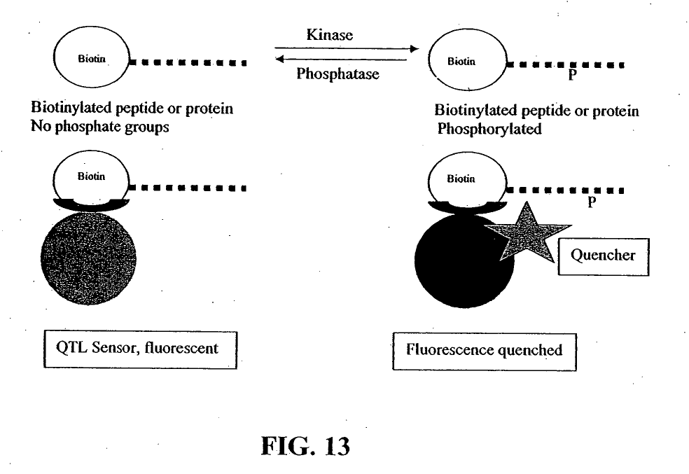

The QT′B format can be used for the detection and quantitation of kinase or phosphatase enzyme activity in a sample. For example, this assay can be used to monitor the phosphorylation or the dephosphorylation, respectively, of biotinylated peptide substrates by target kinases such as PKA and phosphatases such as PTP-1B. The use of a QT′B format for the sensing of kinase or phosphatase activity is shown in FIG. 13.

The QTL sensor can comprise a highly fluorescent conjugated polyelectrolyte co-located with biotin-binding protein, either coated on the surface of a solid support (e.g., a microsphere) as shown in FIG. 13 or present as a complex in solution. A biotinylated peptide or protein substrate that is known to be specifically phosphorylated by a target kinase (e.g., PKA) or dephosphorylated by a target phosphatase (e.g., PTP-1B) can be incubated with the appropriate enzyme for a given time period.

As shown in FIG. 13, a non-phosphorylated BT conjugate can be added to a sample and incubated with the sample to monitor kinase enzyme activity. After incubation of the conjugate with the sample, addition of the polymer sensor and quencher to the sample can result in quenching of polymer fluorescence. The decrease in fluorescence is a linear function of enzymatic activity.

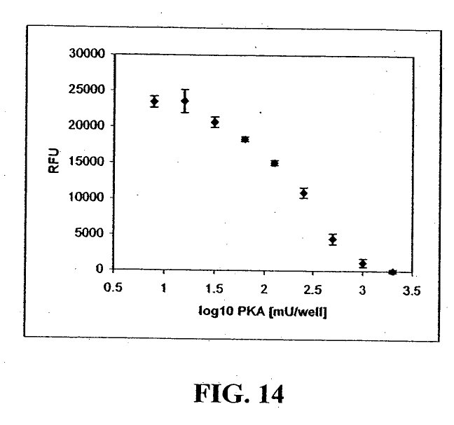

FIG. 14 is a graph showing the measurement of protein kinase A (PKA) activity using a QT′B assay. In FIG. 14, fluorescence (RFU) is plotted as a function of PKA concentration (mU/well). As can be seen from FIG. 14, increasing concentrations of PKA result in decreased fluorescence.

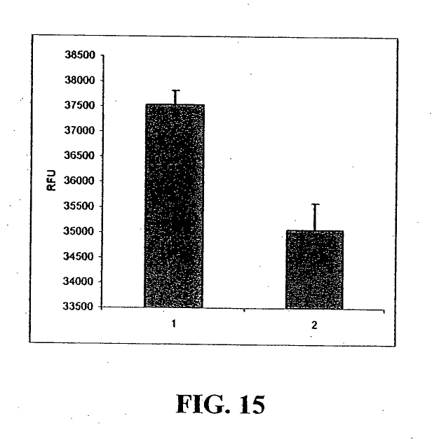

FIG. 15 is a chart illustrating the detection of protein kinase C activity using whole protein substrate, Histone 1. As can be seen from FIG. 15, lower levels of polymer fluorescence are observed for non-phosphorylated histone substrate (2) compared to phosphorylated histone substrate (1).

As also shown in FIG. 13, phosphatase enzyme activity in a sample can be monitored by incubation of the sample with a phosphorylated BT conjugate. The addition of the polymer sensor and quencher to the incubated sample can result in an increase in polymer fluorescence as a function of PTP-1B activity.

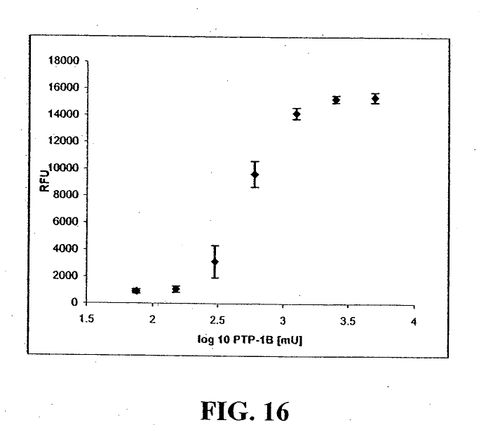

FIG. 16 is a graph illustrating the detection of protein tyrosine phosphatase-1B (PTP-1B) activity using a QT′B assay. In FIG. 16, fluorescence (RFU) is plotted as a function of PTP-1B concentration (mU/well). As can be seen from FIG. 16, increasing concentrations of PTP-1B result in increased fluorescence.

For the detection of PKA kinase activity, a Kemptide peptide substrate can be used. This substrate contains a biotin at the N-terminus and a serine that can be phosphorylated by PKA.

For the detection of PTP-1B phosphatase activity, a phosphorylated substrate with an N-terminal biotin can be used. This substrate can undergo de-phosphorylation upon interaction with PTP-1B.

Unlike FRET (fluorescence resonance energy transfer) assays where the quench is an equimolar event between the donor and acceptor, the QTL kinase and phosphatase assays described above employ a functionally superior platform that combines the well-established phosphate-metal complex interactions with the phenomenon of conjugated polymer superquenching by electron and energy transfer quenchers, resulting in amplification of the fluorescence signal and enhanced sensitivity in the measurement of enzymatic activity.

Metal Ion Mediated Polymer Superquenching Based Bioassays

It has previously been shown that anionic conjugated polymers associate strongly with metal cations and organic cations, sometimes with concurrent quenching of the polymer fluorescence. [1, 4] The association occurs as a consequence of coulombic and hydrophobic interactions. Previous studies have also shown that the association between polymer and counterions can be controlled or tuned by pre-association of the polymer with a charged support such as polystyrene microspheres, silica or clay or with another charged polymer. [4-6]

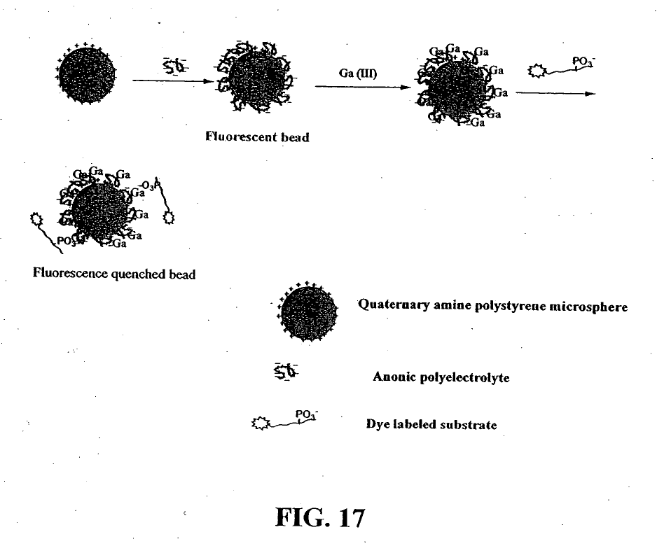

Anionic polymers, an example of which is shown in FIG. 1A, can associate with metal ions in a process which causes little modification of the polymer fluorescence. As an example of this approach, a polymer having the structure shown in FIG. 1A was first coated onto cationic polystyrene microspheres and then treated with Ga³⁺. This process is illustrated in FIG. 17. As can be seen from FIG. 17, the Ga³⁺ associates with the polymer but does not quench its fluorescence. The ensemble consisting of the solid support (e.g., the beads), the polymer and the metal ions (e.g., Ga³⁺) provides a new sensor platform that takes advantage of the previously demonstrated ability of metal ions to associate with organic phosphates.

Metal ion affinity chromatography (IMAC) is a common technique in the purification of phosphorylated species. Metal ions such as Fe(III), Ga(III), Al(III), Zr(IV), Sc(III) and Lu(III) (hard Lewis acids) can be immobilized on the surface of resin beads such as Agarose, Sepharose etc., through association with covalently linked iminodiacetic acetic acid (IDA) or nitrilotriacetic acid (NTA) or other ligands. The bound metal ions can in turn bind to phosphorylated species such as proteins or peptides. In addition to the applications of IMAC in the isolation of proteins, IMAC related technology can be used as a sensing format for protein kinase enzymes by monitoring changes in fluorescence polarization of a fluorescent-labeled substrate upon forming the phosphate metal complex subsequent to phosphorylation.

As shown in FIG. 17, the solid support associated Ga³⁺ retains the ability to complex with phosphorylated substrates generated by kinase enzymes (or dephosphorylated by a phosphatase enzyme). The solid support associated Ga³⁺ can therefore be used to provide the basis for a QTL assay. In the example shown, the substrate has been functionalized with a quencher that can reduce the fluorescence of the fluorescent polymer by either energy or electron transfer quenching when brought into the vicinity of the polymer by association with the metal ion (e.g., Ga³⁺).

An exemplary sensing format employs an anionic polyeletrolyte having a structure as shown in FIG. 1A (hereinafter referred to as “PPE”), a 0.55 μm cationic polystyrene microsphere, gallium chloride, and a rhodamine labeled phosphorylated peptide. This sensing format is illustrated schematically in FIG. 17.

The anionic PPE polymer was first immobilized on the solid support (i.e., 0.55 μm cationic polystyrene microspheres) through deposition in water. The polymer coated microspheres were then treated with gallium chloride in aqueous solution at a pH of 5.5. Excess Ga³⁺ was then washed away.

A dye labeled phosphorylated substance generated from either enzyme phosphorylation reaction (e.g., kinase), protease cleavage reaction, or a single DNA/RNA sequence, or through a competitive reaction may associate with the gallium polymer sensor and modulate the fluorescence from the polymer.

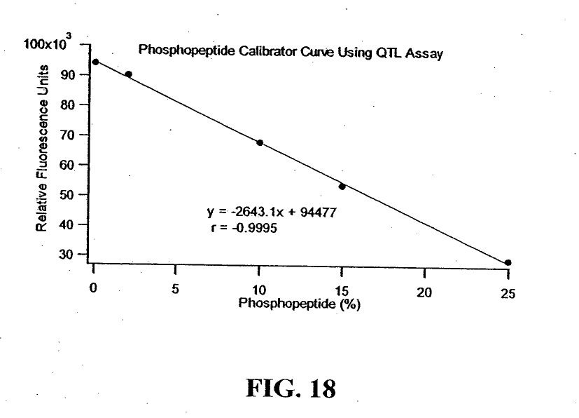

FIG. 18 shows the fluorescence of a gallium polymer sensor as a function of the degree of phosphorylation in a peptide substrate. In FIG. 18, relative fluorecence is plotted as a function of the degree of phosphorylation (% phosphopeptide).

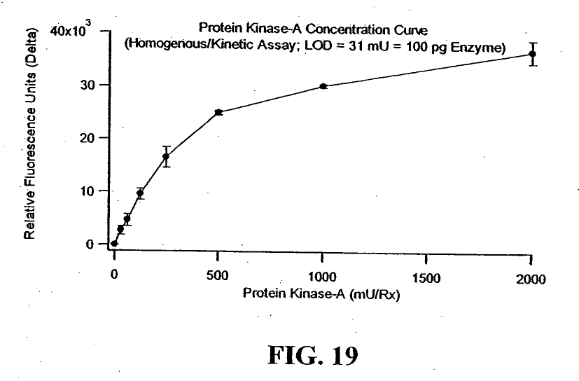

FIG. 19 demonstrates an actual kinetic assay for the level of protein kinase A enzyme in a sample in which the enzyme mediated phosphorylation of the substrate occurs in the presence of the gallium polymer sensor. In FIG. 19, relative fluorecence is plotted as a function of protein kinase A (PKA) concentration (mU/Rx).

The fluorescence change can be monitored in a variety of formats. The general assay may be used to monitor enzyme mediated reactions for a variety of substrates as both a kinetic and end-point assay.

Application of QT′B Sensing Approach to Inhibitor Screening for Drug Discovery

The use of conjugated polymers that exhibit superquenching in the presence of electron or energy transfer quenchers in assays for kinase and phosphatase enzyme activity can be adapted to screen large compound libraries for drugs that alleviate the effects of pharmacologically relevant enzymes and other biomolecules. Addition of a known inhibitor of enzyme activity will interfere with the reaction of enzyme with substrate and thus modulate the signal response otherwise seen in the absence of the inhibitor. The extent of signal modulation seen for a given concentration of the inhibitor is a measure of the strength of the inhibitor.

The QT′B-based assays can be conducted in microtiter plates of various well densities to accelerate the drug discovery process. In one embodiment, a library of compounds can be screened in a kinase or phosphatase assay to look for inhibition of the phosphorylation or dephosphorylation reaction respectively.

Assays, Reagents and Kits Employing a Biotinylated Tether (BT) and a Conjugate of a Quencher and a Biotin Binding Protein

As set forth above, QTL bioconjugates associated with fluorescent polymers have been developed which employ the self-organizing capability of fluorescent polyelectrolytes either as individual molecules in solution or as an assembly on a support to complex with metal ions. The thus complexed metal ions can associate with selectivity to coordinating groups (e.g., phosphate groups) on a bioconjugate comprising a quencher (Q) thus providing the basis for selective detection of proteins, small molecules, peptides, proteases and oligonucleotides.

The approach described above utilizes a bioconjugate which is labeled with a quencher. The bioconjugate, however, can also be assembled in a two-step process wherein a biotinylated substrate is enzymologically reacted in a first step and a detection molecule containing a biotin binding protein molecule (e.g., streptavidin) coupled to a quencher is added in a second step. Upon addition of a sensor, an association of phosphate to metal ion occurs and quench is mediated by the bound biotin binding protein/quencher conjugate.

This “snap-on” approach may also be used in a one-step assay by pre-associating the biotinylated substrate with the streptavidin quencher and using the assembled bioconjugate to react directly with the enzyme. The use of this one-step snap-on assay approach may, however, compromise assay speed and/or sensitivity.

Metal Ion Mediated Superquenching

Conjugated polymers in the poly(phenyleneethynylene) (PPE) family can be prepared with a variety of functional groups appended to the aromatic rings. Among the pendant anionic groups that have been used are those shown schematically in FIG. 1A which shows the molecular structure of a sulfo poly p-phenyleneethynylene (PPE-Di-COOH) conjugated polymer. This polymer can associate with cationic microspheres in water to form a stable polymer coat. The coated microspheres exhibit strong fluorescence. The overall charge on the polymer-coated microspheres can be tuned by the degree of polymer loading and by varying the structure of the polymer.

It has been found that the polymer coated microspheres can associate with metal cations and that the loading of metal cations may depend on the loading level of the polymer on the microsphere. Certain metal ions such as Fe³⁺ and Cu²⁺ can quench the polymer fluorescence while others such as Ga³⁺ do not. Non-quenching metal ions mediate superquenching of microsphere-bound polymer fluorescence under conditions where otherwise, in the absence of the metal ions, little or no quenching would occur. After the polymer-coated microspheres are “charged” by the addition of Ga³⁺, the addition of the phosphorylated peptide to the suspension results in a pronounced quenching of the polymer fluorescence. It was shown that association of the phosphate on the peptide with the Ga³⁺ brings the quencher into close proximity with the polymer and mediates the fluorescence quenching.

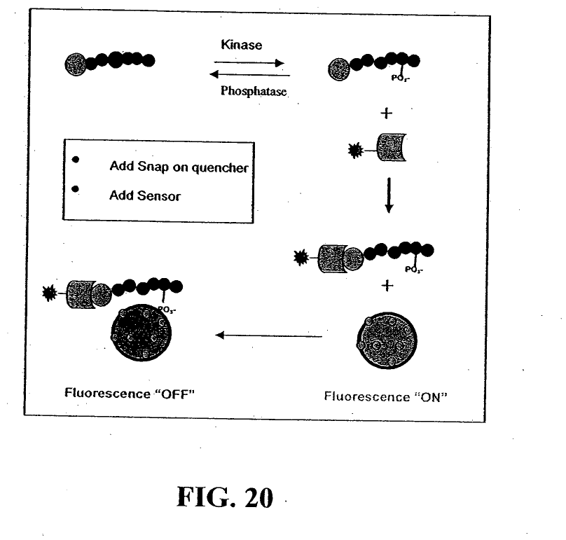

The polymer quench of a phosphorylated biomolecule with the metal ion charged polymer can be achieved in a two-step process is described below. FIG. 20 shows schematically the metal ion mediated superquenching achieved by subsequent addition of a quencher to an enzymatically reacted biotinylated substrate and an example for a kinase assay. FIG. 20 is a schematic illustrating the phosphorylation or dephosphorylation of biotin peptide substrates by target enzymes detected by addition of streptavidin-quencher following QTL sensor. The peptide products are brought to the surface of the polymer by virtue of specific phosphate binding to Ga³⁺ metal ion. The resulting quench of polymer fluorescence is concomitant with phosphorylation or dephosphorylation.

Bioassays Based on Metal Ion Mediated Superquenching—Kinase/Phosphatase Assays

Phosphorylation and dephosphorylation of proteins mediates the regulation of cellular metabolism, growth, differentiation and cell proliferation. Aberration in enzymatic function can lead to diseases such as cancer and inflammation. More than 500 kinases and phosphatases are thought to be involved in the regulation of cellular activity and are possible targets for drug therapy.

Assays exhibiting enhanced sensitivity in the measurement of enzymatic activity by amplifying the fluorescence signal using superquenching have been described. [10-11] The sensor platform used in these assays comprises a modified anionic polyelectrolyte derivative which is immobilized by adsorption on positively charged microspheres. An exemplary modified anionic polyelectrolyte is the derivative of poly(phenyleneethynylene) (PPE) shown in FIG. 1A. Fluorescent polymer superquenching has been adapted to the detection of kinase/phosphatase activity as shown in FIG. 20. Di- or trivalent metal ions can strongly associate with anionic conjugated polymers in solution, resulting in modification and/or quenching of polymer fluorescence. Since the overall charge on a polymer-microsphere ensemble can be tuned, ensembles were constructed to afford a platform whereby metal ions can associate with the polymer without strongly quenching the polymer fluorescence while retaining the ability to complex with specific ligands. For example, it has been found that PPE-associated Ga³⁺ can also associate with phosphorylated peptides such that when the peptide contains a dye such as rhodamine, metal ion mediated polymer superquenching occurs. Here we describe the application of the platform for the detection of biotinylated peptide substrates.

In applications using, for example, scintillation proximity (SPA) or streptavidin membrane supports (SAMs), wash steps are required to separate unbound radioactive ATP or unbound anti-phospho antibodies from the reaction mixture. To retain converted substrate, biotinylated peptides have been used and immobilized via streptavidin or other biotin-binding proteins on various matrixes. As set forth below, metal-ion mediated superquenching can be used to screen the activity of kinases on individual substrates or biotin-peptide libraries. This approach enables researchers to:

1) test substrate specificities of enzyme mutants;

2) evaluate enzyme purity of proprietary enzymes by comparing phosphorylation patterns;

3) monitor for enhanced emission that provides a fluorescence turn-on assay for kinases; and

4) thereby use enhanced emission with appropriate dye-quenchers that shifts detection to the red in order to improve screening of visible auto-fluorescent compounds in libraries.

As an example, streptavidin-coupled fluorescein quenchers can be added to enzymatically reacted biotinylated peptide substrates. This approach provides the basis for sensitive and selective kinase/phosphatase assays as illustrated in FIG. 20. The assays are instantaneous “mix and read” assays which require no wash steps or complex sample preparation.

After incubation of the biotinylated peptide substrate with enzyme in the sample, a conjugate of a quencher and a biotin binding protein (e.g., streptavidin) is added and allowed to associate with the incubated sample (e.g., for 15 minutes at room temperature).

Example 4 below illustrates a robust assay for protein kinase A (PKA) and the comparable performance of the one-step and two-step approaches. In Example 4, the kinase assay functions as a fluorescence “turn off” assay. Since the quencher may exhibit sensitized fluorescence as a consequence of the quenching of polymer fluorescence, the assays can be used as either turn on or turn off, depending on wavelength monitored. Further, monitoring simultaneously the fluorescence of the polymer and quencher provides for a sensitive ratiometric assay.

### EXAMPLE 4

### Assays for Protein Kinase A (PKA) Activity

The peptides used as enzyme substrates and as phospho-peptide calibrators are described below. For detection of PKA activity in a one-step mode,

| | |
|---|---|
| Rhodamine-LRRASLG | SEQ ID NO:2 |

and the calibrator peptide

| | |
|---|---|
| Rhodamine-LRRA(pS)LG | SEQ ID NO:1 |

were synthesized by Anaspec.

For detection of PKA activity in a two-step mode

| | |
|---|---|
| biotin-LRRASLG | SEQ ID NO:5 |
| and | |
| biotin-LRRA(pS)LG | SEQ ID NO:6 |

were purchased from Anaspec. Recombinant PKA was purchased from Promega. Streptavidin-coupled fluorescein was obtained from Molecular Probes. Polystyrene functionalized beads were obtained from Interfacial Dynamics.

The performance of the one-step versus the two-step approach was determined by reacting 1 μM peptide (either Rhodamine-peptide or biotin-peptide) in assay buffer for 60 minutes at CRT. For the two-step process 5 μL of streptavidin-fluorescein was added and incubated for 15 minutes at CRT. Lastly, 15 μL of sensor in detector buffer were added. The fluorescence of the mixture was measured using a SpectraMax Gemini XS plate reader (Molecular Devices, Inc.) in well scan mode and with excitation at 450 nm with a 475 nm cutoff filter and emission at 490 nm.

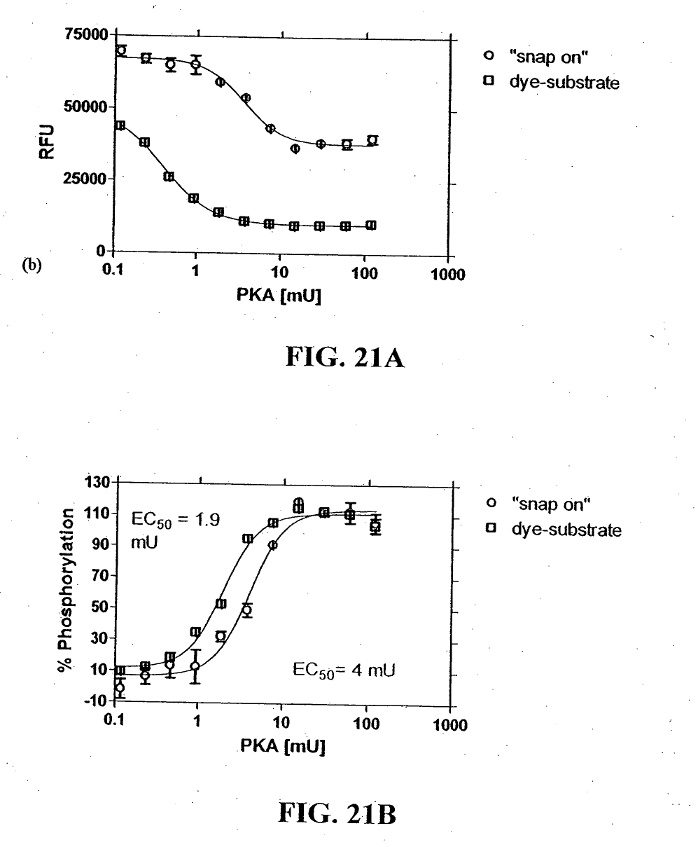

As shown in FIGS. 21A and 21B, the assays perform using either synthetic substrates with an N-terminal quencher or using biotinylated substrates to which a streptavidin-fluorescein conjugate is added. Upon phosphorylation of the substrate, the peptide associates to the sensor via the phosphate groups and quenches the fluorescence.

FIGS. 21A and 21B are graphs showing an enzyme concentration curve for PKA using rhodamine-labeled substrates or biotinylated substrates in a two step approach. The RFU generated in the assays are shown in FIG. 21A and the % Phosphorylation following backcalculation from a standard curve are shown in FIG. 21B. In FIGS. 21A and 21B, a concentration of 1 μM substrate was phosphorylated using serially diluted kinase PKA enzyme for 1 hour at room temperature in a white 384-well Optiplate. Following incubation, 5 μmol streptavidin-rhodamine conjugate was added and incubated for 15 minutes at approximately 22° C. followed by the addition of approximately 100×10⁶ QTL Sensor beads and incubation for 10 minutes at approximately 22° C. Plates were incubated for 30 minutes at approximately 22° C. and the fluorescence signal monitored using excitation at 450 nm, emission at 490 nm with a 475 nm cutoff filter in a Gemini XS Plate reader (Molecular Devices, Inc.).

### EXAMPLE 5

### Assays for Screening Substrates for PKA, PKCa or PTP-1B

For substrate screening, 1 μM biotin-peptide was reacted in assay buffer for 60 minutes at approximately 22° C. Control reactions contained no enzyme. Subsequently 5 μL of streptavidin-fluorescein conjugate was added and incubated for 15 minutes at approximately 22° C. Lastly, 15 μL of sensor in detector buffer was added. The fluorescence of the mixture was measured using a SpectraMax Gemini XS plate reader (Molecular Devices, Inc.) in well scan mode and with excitation at 450 nm with a 475 nm cutoff filter and emission at 490 nm.

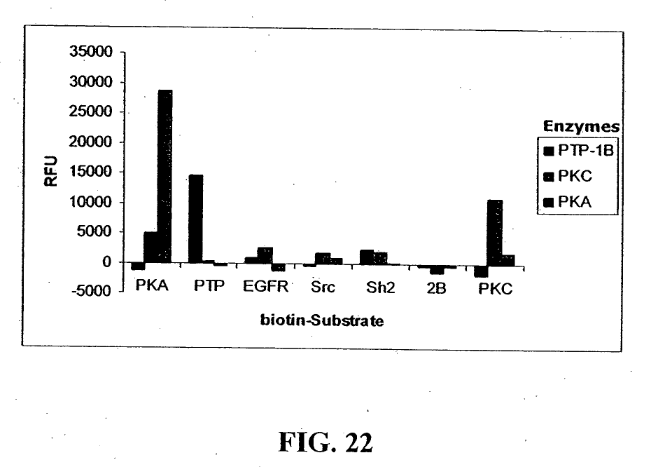

FIG. 22 is a bar chart illustrating the screening of seven (7) different biotinylated substrates for kinase or phosphatase with enzymes PTP-1B, PKCα and PKA. Reactions were run with or without enzyme and the difference in RFU was computed and plotted. As can be seen from FIG. 22, phosphorylation dependent quench of fluorescence was detected only in reactions containing the appropriate substrate and not in reactions containing nonspecific substrates.

According to one embodiment, the quenching sensitivity of the amplified superquenching as measured by the Stem-Volmer quenching constant is at least 500. According to further embodiments, the quenching sensitivity of the amplified superquenching as measured by the Stern-Volmer quenching constant is at least 1000, 2000, 5000, 10,000, 100,000 or 1×10⁶.

Exemplary fluorescers include fluorescent polymers. Exemplary fluorescent polymers include luminescent conjugated materials such as, for example, a poly(phenylene vinylene) such as poly(p-phenylene vinylene) (PPV), polythiophene, polyphenylene, polydiacetylene, polyacetylene, poly(p-naphthalene vinylene), poly(2,5-pyridyl vinylene) and derivatives thereof such as poly(2,5-methoxy propyloxysulfonate phenylene vinylene) (MPS-PPV), poly(2,5-methoxy butyloxysulfonate phenylene vinylene) (MBS-PPV) and the like. For water solubility, derivatives can include one or more pendant ionic groups such as sulfonate and methyl ammonium. Exemplary pendant groups include:
—O—(CH₂)ₙ—OSO₃⁻(M⁺)
wherein n is an integer (e.g., n=3 or 4) and M⁺ is a cation (e.g., Na⁺ or Li⁺);
—(CH₂)ₙ—OSO₃⁻(M⁺)
where n is an integer (e.g., n=3 or 4) and M⁺ is a cation (e.g., Na⁺ or Li⁺);
—O—(CH₂)ₙ—N⁺(CH₃)₃(X⁻)
where n is an integer (e.g., n=3 or 4) and X⁻ is an anion (e.g., Cl); and
—(CH₂)ₙ—N⁺(CH₃)₃(X⁻)
where n is an integer (e.g., n=3 or 4) and X⁻ is an anion (e.g., Cl⁻).

While the foregoing specification teaches the principles of the present application, with examples provided for the purpose of illustration, it will be appreciated by one skilled in the art from reading this disclosure that various changes in form and detail can be made without departing from the true scope of the disclosure.

### CITED REFERENCES

[1] Chen, L. et al, Proc. Natl. Acad. Sci. 1999, 96, 12287.
[2] Chen, L. et al, Chem. Phys. Lett. 2000, 330, 27.
[3] Chen, L. et al, J. Am. Chem. Soc. 2000, 122, 9302.
[4] Jones, R. M. et al, Langmuir 2000, 17, 2568.
[5] Jones, R. M. et al, J. Am. Chem. Soc. 2001, 123, 6726.
[6] Jones, R. M. et al, Proc. Natl. Acad. Sci. 2001, 98, 14769.
[7] Kushon, S. A. et al, Langmuir 2002, 18, 7245.
[8] Lu, L. et al, J. Am. Chem. Soc. 2002, 124, 483.
[9] Kumaraswamy, S. et al, Proc. Natl. Acad. Sci. 2004, 101, 7511.
[10] Xia, W. et al, Assay and Drug Dev. Techn., 2004, 2, 183
[11] Xia, W. et al, American Laboratory, 2004, 36, 15.

### OTHER REFERENCES

Zhou, W. et al, J. Am. Soc. Mass Spectrom 2000, 273.
Breuer, W. et al, J. Biol. Chem. 1995 270, 24209.
Rininsland et al., Proc. Natl. Acad. Sci. 2004, 101, 15295.

What is claimed is:

1. A complex comprising:
a biotinylated polypeptide, wherein the polypeptide comprises one or more phosphate groups; and
a metal cation associated with a phosphate group of the polypeptide.

2. The complex of claim 1, wherein the metal cation is Ga³⁺.

3. The complex of claim 1, further comprising a fluorescer;
wherein the fluorescer comprises one or more anionic groups and a plurality of fluorescent species associated with one another such that a quencher is capable of amplified superquenching of the fluorescer when the quencher is associated with the fluorescer, wherein the fluorescer is associated with a biotin binding protein; and
wherein an anionic group of the fluorescer is associated with the metal cation.

4. The complex of claim 3, wherein the fluorescer is a fluorescent polymer.

5. The complex of claim 3, wherein the fluorescer is a poly(p-phenylene-ethynylene) polymer.

6. The complex of claim 3, wherein the fluorescer is associated with the surface of a solid support.

7. The complex of claim 6, wherein the solid support is a microsphere.

8. The complex of claim 6, wherein the solid support comprises a positively charged surface and wherein an anionic group of the fluorescer is associated with the positively charged surface.

9. The complex of claim 3, further comprising a quencher capable of amplified super-quenching of the fluorescer when associated therewith, wherein the quencher is associated with a phosphate group of the polypeptide.

10. The complex of claim 9, wherein the quencher is an organometallic compound.

11. The complex of claim 10, wherein the quencher is an iron(III) iminodiacetic acid chelate.

12. The complex of claim 3, wherein the fluorescer and the biotin binding protein are associated with the surface of a solid support.

13. A method of detecting the presence and/or amount of a kinase or phosphatase enzyme analyte in a sample, the method comprising:
a) incubating the sample with a biotinylated polypeptide, wherein, for a kinase enzyme analyte, the polypeptide comprises one or more groups which are phosphorylatable by the analyte or, wherein for a phosphatase enzyme analyte, the polypeptide comprises one or more groups which are dephosphorylatable by the analyte;
b) adding to the sample a metal cation, wherein either the metal cation is a quencher or wherein the method further comprises adding to the sample a quencher which can associate with the metal cation;
c) adding to the sample a fluorescer comprising a plurality of fluorescent species associated with one another such that the quencher is capable of amplified superquenching of the fluorescer when the quencher is associated with the fluorescer, wherein the fluorescer is associated with a biotin binding protein;
d) detecting fluorescence;
wherein the detected fluorescence indicates the presence and/or amount of analyte in the sample.

14. The method of claim 13, wherein the quencher associates with the phosphorylated polypeptide.

15. The method of claim 14, wherein the polypeptide comprises groups which are phosphorylatable by the analyte; and
wherein phosphorylation of the phosphorylatable groups results in a decrease in fluorescence.

16. The method of claim 14, wherein the polypeptide comprises groups which are dephosphorylatable by the analyte; and
wherein dephosphorylation of the groups results in an increase in fluorescence.

17. The method of claim 13, wherein the metal cation is Ga³⁺.

18. The method of claim 13, wherein the fluorescer is a fluorescent polymer.

19. The method of claim 18, wherein the fluorescer is a poly(p-phenylene-ethynylene) polymer.

20. The method of claim 13, wherein the fluorescer is associated with the surface of a solid support.

21. The method of claim 13, wherein the fluorescer and the biotin binding protein are associated with the surface of a solid support.

22. The method of claim 20, wherein the solid support is a microsphere.

23. The method of claim 20, wherein the solid support comprises a positively charged surface;
wherein the fluorescer comprises one or more anionic groups; and
wherein an anionic group of the fluorescer is associated with the positively charged surface.

24. The method of claim 13, wherein the quencher is an organometallic compound.

25. The method of claim 14, wherein the quencher is an iron(III) iminodiacetic acid chelate.

26. The method of claim 13, wherein the fluorescer, the quencher, and the metal cation are added to the sample after incubation and before detecting fluorescence.

27. The method of claim 13, wherein the fluorescer, the quencher, and the metal cation are added to the sample before incubation or during incubation and wherein detecting fluorescence comprises detecting fluorescence during incubation.

28. A method of screening a compound as an inhibitor of kinase or phosphatase enzyme activity comprising:
a) incubating in a sample a biotinylated polypeptide with a kinase or phosphatase enzyme in the presence of the compound, wherein, for a kinase enzyme assay, the polypeptide comprises one or more groups which are phosphorylatable by the analyte and wherein, for a phosphatase enzyme assay, the polypeptide comprises one or more groups which are dephosphorylatable by the analyte;
b) adding to the sample a metal cation, wherein either the metal cation is a quencher or wherein the method further comprises adding to the sample a quencher which can associate with the metal cation;
c) adding to the sample a fluorescer comprising a plurality of fluorescent species associated with one another such that the quencher is capable of amplified superquenching of the fluorescer when the quencher is associated with the fluorescer, wherein the fluorescer is associated with a biotin binding protein; and
d) detecting fluorescence from the sample in the presence of the compound;
wherein the amount of fluorescence detected in the presence of the compound indicates the inhibitory effect of the compound on kinase or phosphatase enzyme activity.

29. The method of claim 28, further comprising:
a) incubating in a second sample the biotinylated polypeptide with the kinase or phosphatase enzyme in the presence of a second compound;
b) adding to the second sample the fluorescer, the quencher, and the metal cation;
c) detecting fluorescence from the second sample in the presence of the second compound;
wherein the amount of fluorescence detected from the second sample indicates the inhibitory effect of the second compound on kinase or phosphatase enzyme activity.

30. The method of claim 28, further comprising:
a) incubating in a second sample the biotinylated polypeptide with the kinase or phosphatase enzyme, wherein the second sample is devoid of the compound;
b) adding to the second sample the fluorescer, the quencher, and the metal cation; and
c) detecting fluorescence from the second sample in the absence of the compound;
wherein the amount of fluorescence detected from the second sample in the absence of the compound is the baseline fluorescence.

31. The method of claim 30, further comprising:
comparing the fluorescence detected in the presence of the compound to the baseline fluorescence detected in the absence of the compound;
wherein a difference in the fluorescence detected in the presence of the compound and the baseline fluorescence is an indication of the inhibitory effect of the compound on kinase or phosphatase enzyme activity.

32. A bioconjugate comprising:
a polypeptide comprising one or more phosphorylatable or dephosphorylatable groups; and
a quenching moiety conjugated to the polypeptide, wherein the quenching moiety is capable of amplified super-quenching of a fluorescent polymer when associated therewith.

33. The bioconjugate of claim 32, wherein the quenching moiety is rhodamine.

34. The bioconjugate of claim 32, wherein the polypeptide comprises one or more phosphate groups.

35. The bioconjugate of claim 34, wherein the polypeptide further comprises a cleavage site and wherein the quenching moiety and the phosphate groups are on opposite sides of the cleavage site and wherein no phosphate groups are present on the side of the cleavage site to which the quenching moiety is conjugated.

36. The bioconjugate of claim 34, wherein the polypeptide further comprises a cleavage site and wherein the quenching moiety and the phosphate groups are on the same side of the cleavage site and wherein no phosphate groups are present on the side of the cleavage site opposite the side to which the quenching moiety is conjugated.

37. A method of detecting the presence and/or amount of a protease enzyme in a sample, the method comprising:
a) incubating the sample with a bioconjugate as set forth in claim 35 wherein the protease enzyme cleaves the polypeptide at the cleavage site;
b) adding to the sample a fluorescer comprising a plurality of fluorescent species associated with one another such that the quenching moiety is capable of amplified superquenching of the fluorescer when the quenching moiety is associated with the fluorescer, wherein the fluorescer further comprises one or more anionic groups and wherein at least one metal cation is associated with an anionic group of the fluorescer; and
c) detecting fluorescence from the sample;
wherein the detected fluorescence indicates the presence and/or amount of protease enzyme in the sample.

38. A kit for detecting the presence and/or amount of a kinase or phosphatase enzyme analyte in a sample comprising:
a first component comprising a bioconjugate as set forth in claim 32; and
a second component comprising a fluorescer, the fluorescer comprising a plurality of fluorescent species associated with one another such that the quenching moiety of the bioconjugate is capable of amplified superquenching of the fluorescer when the quenching moiety is associated with the fluorescer, wherein the fluorescer further comprises one or more anionic groups and wherein at least one metal cation is associated with an anionic group of the fluorescer.

39. The kit of claim 38, wherein the fluorescer is a fluorescent polymer.

40. The kit of claim 38, wherein the fluorescer is a poly(p-phenylene-ethynylene) polymer.

41. The kit of claim 38, wherein the fluorescer is associated with the surface of a solid support.

42. The kit of claim 41, wherein the solid support is a microsphere.

43. The kit of claim 41, wherein the solid support comprises a positively charged surface and wherein one or more anionic groups of the fluorescer are associated with the positively charged surface.

44. The kit of claim 38, wherein the quenching moiety is rhodamine.

45. A method of detecting the presence and/or amount of an enzyme analyte in a sample, the method comprising:
a) incubating the sample with a bioconjugate as set forth in claim 32, wherein the polypeptide of the bioconjugate comprises groups which are phosphorylatable or dephosphorylatable by the enzyme analyte;
b) adding to the sample a fluorescer comprising a plurality of fluorescent species associated with one another such that the quenching moiety is capable of amplified superquenching of the fluorescer when the quenching moiety is associated with the fluorescer, wherein the fluorescer further comprises one or more anionic groups and wherein at least one metal cation is associated with an anionic group of the fluorescer; and
c) detecting fluorescence from the sample;
wherein the detected fluorescence indicates the presence and/or amount of analyte in the sample.

46. The method of claim 45, wherein the polypeptide comprises groups which are phosphorylatable by the analyte and wherein phosphorylation of the phosphorylatable groups of the polypeptide results in a decrease in fluorescence.

47. The method of claim 45, wherein the polypeptide comprises groups which are dephosphorylatable by the analyte and wherein dephosphorylation of the dephosphorylatable groups of the polypeptide results in an increase in fluorescence.

48. The method of claim 45, wherein the metal cation is Ga³⁺.

49. The method of claim 45, wherein the fluorescer is a fluorescent polymer.

50. The method of claim 49, wherein the fluorescer is a poly(p-phenylene-ethynylene) comprising anionic groups.

51. The method of claim 45, wherein the fluorescer is associated with the surface of a solid support.

52. The method of claim 51, wherein the solid support is a microsphere.

53. The method of claim 51, wherein the solid support comprises a positively charged surface and wherein an anionic group of the fluorescent polymer is associated with the positively charged surface.

54. The method of claim 45, wherein the fluorescer is added to the sample after incubation and before detecting fluorescence.

55. The method of claim 45, wherein the fluorescer is added to the sample before incubation or during incubation and wherein detecting fluorescence comprises detecting fluorescence during incubation.

56. A kit for detecting the presence of an analyte in a sample comprising:
a first component comprising a quencher; and
a second component comprising a biotinylated polypeptide, wherein the polypeptide can be modified by the analyte and wherein the polypeptide modified by the analyte associates with the quencher.

57. The kit of claim 56, further comprising a fluorescer comprising a plurality of fluorescent species associated with one another such that the quencher is capable of amplified super-quenching of the fluorescer when associated therewith.

58. The kit of claim 57, wherein the fluorescer is a fluorescent polymer.

59. The kit of claim 57, wherein the fluorescent polymer is a poly(p-phenylene-ethynylene) polymer.

60. The kit of claim 57, wherein the fluorescer is associated with the surface of a solid support.

61. The kit of claim 60, wherein the solid support is a microsphere.

62. The kit of claim 56, wherein the analyte is an enzyme.

63. The kit of claim 62, wherein the enzyme is a kinase or phosphatase enzyme.

64. The kit of claim 62, wherein the enzyme can phosphorylate the polypeptide substrate and wherein the phosphorylated peptide substrate associates with the quencher.

65. The kit of claim 56, wherein the quencher is an organometallic compound.

66. The kit of claim 56, wherein the quencher is an iron(III) iminodiacetic acid chelate.

67. A method of detecting the presence and/or amount of a phosphodiesterase enzyme in a sample, the assay comprising:
a) incubating the sample with a bioconjugate comprising a quencher conjugated to cyclic AMP or cyclic GMP;
b) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and
c) detecting fluorescence from the sample;
wherein the amount of detected fluorescence indicates the presence and/or amount of phosphodiesterase enzyme in the sample.

68. The method of claim 67, wherein the fluorescer and the metal cation are added to the sample after incubation and before detecting fluorescence.

69. The method of claim 67, wherein the fluorescer and the metal cation are added to the sample before incubation or during incubation and wherein detecting fluorescence comprises detecting fluorescence during incubation.

70. A method of detecting kinase enzyme activity of a polypeptide substrate, the method comprising:
a) incubating the polypeptide substrate and a quencher labeled polypeptide comprising one or more phosphorylatable groups with a sample comprising a kinase enzyme;
b) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups, and wherein at least one metal cation is associated with an anionic group of the fluorescer; and
c) detecting fluorescence from the sample;
wherein phosphorylation of the polypeptide substrate results in an increase in fluorescence; and
wherein the amount of fluorescence detected indicates the presence and/or amount of kinase enzyme activity of the polypeptide substrate.

71. The method of claim 70, wherein the polypeptide substrate is a natural protein.

72. The method of claim 70, wherein the fluorescer and the metal cation are added to the sample after incubation and before detecting fluorescence.

73. The method of claim 70, wherein the fluorescer and the metal cation are added to the sample before incubation or during incubation and wherein detecting fluorescence comprises detecting fluorescence during incubation.

74. A method of detecting the presence and/or amount of a nucleic acid analyte in a sample, the assay comprising:
a) incubating the sample with a polynucleotide comprising a quencher conjugated to the polypeptide in a first terminal region of the polynucleotide and a phosphate group in a second terminal region of the polynucleotide, wherein at least a portion of the first and second terminal regions of the polynucleotide can hybridize together to form a hairpin structure and wherein a central region of the polynucleotide between the terminal regions comprises a nucleic acid sequence which can hybridize to the nucleic acid analyte thereby disrupting the hairpin structure and resulting in separation of the quencher and the phosphate group of the polynucleotide;
b) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and
c) detecting fluorescence from the sample;
wherein the detected fluorescence indicates the presence and/or amount of nucleic acid analyte in the sample.

75. A method of detecting the presence and/or amount of a nucleic acid analyte in a sample, the assay comprising:
a) labeling nucleic acids in the sample with a quencher;
b) incubating the sample with a polynucleotide comprising a phosphate group in a first terminal region of the polynucleotide, wherein the polynucleotide comprises a nucleic acid sequence which can hybridize to the nucleic acid analyte;
c) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and
d) detecting fluorescence from the sample;
wherein hybridization of the nucleic acid analyte to the polynucleotide results in a decrease in fluorescence; and
wherein decreased fluorescence indicates the presence and/or amount of nucleic acid analyte in the sample.

76. A method of detecting the presence and/or amount of a nucleic acid analyte in a sample, the method comprising:
a) incubating the sample with a first polynucleotide comprising a phosphate group in a terminal region thereof and a second polynucleotide comprising a quencher conjugated to the second polynucleotide in a terminal region thereof, wherein the second polynucleotide and the nucleic acid analyte can hybridize to the first polynucleotide;
b) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and
c) detecting fluorescence from the sample;
wherein hybridization of the nucleic acid analyte to the first polynucleotide results in an increase in fluorescence; and
wherein the amount of fluorescence detected indicates the presence and/or amount of nucleic acid analyte in the sample.

77. The method of claim 76, wherein the phosphate group is in a 3′-terminal region of the first polynucleotide and the quencher is in a 5′-terminal region of the second polynucleotide or wherein the phosphate group is in a 5′-terminal region of the first polynucleotide and the quencher is in a 3′-terminal region of the second polynucleotide.

78. A method of detecting the presence and/or amount of a polypeptide analyte in a sample, the assay comprising:
a) incubating the sample with: a nucleic acid aptamer comprising a phosphate group in a terminal region thereof, wherein the nucleic acid aptamer can bind to the polypeptide analyte; and a polynucleotide comprising a quencher, wherein the polynucleotide can hybridize to the nucleic acid aptamer;
b) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and
c) detecting fluorescence from the sample;
wherein binding of the polypeptide analyte to the nucleic acid aptamer results in an increase in fluorescence; and
wherein the amount of fluorescence detected indicates the presence and/or amount of polypeptide analyte in the sample.

79. The method of claim 78, wherein the phosphate group is in a 3′-terminal region of the nucleic acid aptamer and the quencher is in a 5′-terminal region of the polynucleotide or wherein the phosphate group is in a 5′-terminal region of the nucleic acid aptamer and the quencher is in a 3′-terminal region of the polynucleotide.

80. The method of claim 78, wherein the polypeptide analyte is a natural protein.

81. A complex comprising:
a polypeptide comprising a biotin moiety wherein one or more amino acid residues of the polypeptide are phosphorylatable or dephosphorylatable; and
a biotin binding protein conjugated to a quenching moiety;
wherein the biotin moiety of the polypeptide is associated with the biotin binding protein via protein-protein interactions; and
wherein the quenching moiety is capable of amplified super-quenching of a fluorescer when associated therewith.

82. The complex of claim 81, wherein the polypeptide comprises one or more phosphate groups.

83. The complex of claim 82, further comprising a metal cation associated with a phosphate group of the polypeptide.

84. The complex of claim 83, wherein the metal cation is Ga³⁺.

85. The complex of claim 83, further comprising a fluorescer;
wherein the fluorescer comprises one or more anionic groups and a plurality of fluorescent species associated with one another such that the quencher is capable of amplified superquenching of the fluorescer when the quencher is associated with the fluorescer; and
wherein an anionic group of the fluorescer is associated with the metal cation.

86. The complex of claim 85, wherein the fluorescer is a fluorescent polymer.

87. The complex of claim 85, wherein the fluorescer is a poly(p-phenylene-ethynylene) polymer.

88. The complex of claim 85, wherein the fluorescer is associated with the surface of a solid support.

89. The complex of claim 88, wherein the solid support is a microsphere.

90. The complex of claim 88, wherein the solid support comprises a positively charged surface and wherein an anionic group of the fluorescer is associated with the positively charged surface.

91. The complex of claim 81, wherein the biotin binding protein is streptavidin.

92. The complex of claim 81, wherein the quenching moiety is fluorescein.

93. A method of detecting the presence and/or amount of a kinase or phosphatase enzyme analyte in a sample, the method comprising:
a) incubating the sample with a complex as set forth in claim 81, wherein for a kinase enzyme analyte, the polypeptide comprises one or more groups which are phosphorylatable by the analyte and, wherein for a phosphatase enzyme analyte, the polypeptide comprises one or more groups which are dephosphorylatable by the analyte;
b) adding to the sample a fluorescer comprising a plurality of fluorescent species associated with one another such that the quencher is capable of amplified superquenching of the fluorescer when the quencher is associated with the fluorescer, wherein the fluorescer further comprises one or more anionic groups and wherein at least one metal cation is associated with an anionic group of the fluorescer; and
c) detecting fluorescence from the sample;
wherein the amount of fluorescence detected indicates the presence and/or amount of analyte in the sample.

94. The method of claim 93, wherein the fluorescer and the metal cation are added to the sample after incubation and before detecting fluorescence.

95. The method of claim 93, wherein the fluorescer and the metal cation are added to the sample before incubation or during incubation and wherein detecting fluorescence comprises detecting fluorescence during incubation.

96. A method of detecting the presence and/or amount of a kinase or phosphatase enzyme analyte in a sample, the method comprising:
a) incubating the sample with a biotinylated polypeptide comprising either one or more groups which are phosphorylatable by the analyte for a kinase enzyme analyte assay or one or more groups which are dephosphorylatable by the analyte for a phosphatase enzyme analyte assay;
b) adding to the incubated sample a biotin binding protein conjugated to a quenching moiety;
c) adding to the sample a fluorescer comprising a plurality of fluorescent species associated with one another such that the quenching moiety is capable of amplified superquenching of the fluorescer when the quenching moiety is associated with the fluorescer, wherein the fluorescer further comprises one or more anionic groups and wherein at least one metal cation is associated with an anionic group of the fluorescer; and
d) detecting fluorescence from the sample;
wherein the detected fluorescence indicates the presence and/or amount of analyte in the sample.

97. The method of claim 96, wherein the fluorescer and the metal cation are added to the sample after incubation and before detecting fluorescence.

98. The method of claim 96, wherein the fluorescer and the metal cation are added to the sample before incubation or during incubation and wherein detecting fluorescence comprises detecting fluorescence during incubation.


SEQUENCE LISTING (6 sequences)

SEQ ID NO: 1 - protein, 7 aa, Artificial Sequence
  Description of Artificial Sequence Synthetic peptide
  MOD_RES (1): Rhodamine-Leu
  MOD_RES (5): Phosphorylated-Ser
LRRASLG

SEQ ID NO: 2 - protein, 7 aa, Artificial Sequence
  Description of Artificial Sequence Synthetic peptide
  MOD_RES (1): Rhodamine-Leu
LRRASLG

SEQ ID NO: 3 - protein, 15 aa, Artificial Sequence
  Description of Artificial Sequence Synthetic peptide
  MOD_RES (1): Rhodamine-Lys
  MOD_RES (10): Phosphorylated-Tyr
KVEKIGEGTYGVVYK

SEQ ID NO: 4 - protein, 15 aa, Artificial Sequence
  Description of Artificial Sequence Synthetic peptide
  MOD_RES (1): Rhodamine-Lys
KVEKIGEGTYGVVYK

SEQ ID NO: 5 - protein, 7 aa, Artificial Sequence
  Description of Artificial Sequence Synthetic peptide
  MOD_RES (1): Biotin-Leu
LRRASLG

SEQ ID NO: 6 - protein, 7 aa, Artificial Sequence
  Description of Artificial Sequence Synthetic peptide
  MOD_RES (1): Biotin-Leu
  MOD_RES (5): Phosphorylated-Ser
LRRASLG